(19) United States
(12) Patent Application Publication
(10) Pub. No.: US 2020/0082925 A1
(43) Pub. Date: Mar. 12, 2020

(54) NON-TRANSITORY COMPUTER READABLE RECORDING MEDIUM STORING ALLERGY PRESCRIPTION SEARCH PROGRAM

(71) Applicant: JAPAN NATURAL AGING CARE LAB. CO., LTD., Tokyo (JP)

(72) Inventor: Jun IWATA, Tokyo (JP)

(73) Assignee: JAPAN NATURAL AGING CARE LAB. CO., LTD., Tokyo (JP)

(21) Appl. No.: 16/684,513

(22) Filed: Nov. 14, 2019

Related U.S. Application Data
(63) Continuation of application No. PCT/JP2018/013599, filed on Mar. 30, 2018.

(30) Foreign Application Priority Data
May 24, 2017 (JP) 2017-102590
Dec. 21, 2017 (JP) 2017-245389

(51) Int. Cl. G16H 20/10; G16H 50/20; G06N 3/02

(52) U.S. Cl. CPC G16H 20/10; G06N 3/02; G16H 50/20

(57) ABSTRACT

According to one embodiment, a prescription search method includes an input step of inputting an actual symptom of the pollinosis and a search step of referring to three or more levels of first degree of associations between each symptom of the pollinosis and a prescription for the symptom stored in a database to search for one or more prescriptions based on the symptom input in the input step. The database is configured such that when the database newly obtains a relation between each of the symptoms of the pollinosis and the prescription for the symptom, the database reflects the relation to the first degree of association to update the first degree of association.

### CROSS-REFERENCE TO RELATED APPLICATIONS

This application is a Continuation Application of PCT application No. PCT/JP2018/13599, filed on Mar. 30, 2018, and is based upon and claims the benefit of both of priority from Japanese Patent Application No. 2017-102590, filed on May 24, 2017, and Japanese Patent Application No. 2017-245389, filed on Dec. 21, 2017, the entire contents of which are incorporated herein by reference.

### FIELD

The present invention relates to non-transitory computer-readable recording medium storing allergy prescription search program preferred to automatically search for an optimal prescription from a symptom of a patient with allergy, such as pollinosis.

### BACKGROUND

Pollinosis, which is an allergic disease common and specific to Japan, is caused by pollen of a plant, such as a Japanese cedar, in contact with a mucous membrane, such as a nose and eyes. Among them, the pollen of the Japanese cedar has a grain diameter of around 20 μm to 40 μm, and an attachment of the pollen to the mucous membrane of the nose causes a symptom, such as sternutation, a nasal discharge, and stuffy nose. Additionally, pollen entering the eyes causes a symptom, such as itchy eyes. These respective symptoms of pollinosis cause a patient to suffer from a significant discomfort over a long period of time, thus giving a host of negative effects to daily life, a job, and the like.

In view of this, various medical agents to reduce the symptom of pollinosis have been conventionally developed (for example, see JP-A-2014-172842 and JP-A-2011-162544). Additionally, various masks to avoid taking pollen have been also developed (for example, see JP-A-2006-055320).

However, all of these medical agents do not have a nature to thoroughly cure pollinosis through ingestion but merely temporarily reduce the symptoms. Accordingly, each time that reducing effects provided by the medical agent become less effective, the patient continues ingesting the medical agent. Moreover, the mask merely blocks the taking of pollen physically; therefore, this does not lead to the thorough cure of pollinosis.

Generally, an amount of scatter of the pollen of the Japanese cedar increases in March and April; however, ingesting the medical agent to reduce the symptom continuously or keep wearing the mask in this period places a heavy burden on the patient. Additionally, even when the season when the pollen scatters ends, since the pollinosis is not thoroughly cured originally, whenever the season comes every year, the medical agent to reduce the symptom needs to be continuously ingested or the mask needs to be worn continuously.

It is estimated that the number of patients with pollinosis is around thirty million people in Japan. A wish of the patients is the thorough cure for pollinosis, not the temporal reduction of pollinosis and just the physical block of pollen. For the thorough cure for pollinosis, it is important to improve a physical constitution and lifestyle habits themselves by prescription, such as meals, supplements, exercises, and sleeping, without relying on the medical agents.

Attempts to provide optimal prescriptions for various diseases, such as diabetes, cancer, and high blood pressure, and various symptoms, such as thin hair, have been conventionally variously studied. Attempts to provide optimal prescriptions for various symptoms affecting a beauty care, such as a blotch and acne on a face, a flexion, and a rough skin, have been conventionally variously studied.

The optimal prescriptions against these various symptoms are greatly affected by ingesta based on eating habits of the patient and attribute information, such as an age, a sex, and heredity of the patient. In view of this, to propose the optimal prescription for each patient, it is necessary to detect the ingesta and the attribute information of each patient and provide the optimal prescription considering a relation between the detected ingesta and attribute information.

However, the respective ingesta and attribute information of the patient are varied in wide range, and combinations of these reach several million patterns, reaching an enormous volume of patterns. Thus, manually searching the optimal prescriptions for the individual ingesta and attribute information, and the combinations of them by manpower has been actually extremely difficult.

A medical assistance system that checks an interaction brought by a prescription medicine with respect to the attribute information of the patient has been conventionally disclosed. There has been also proposed a technique that quantifies adaptability of a prescription for a symptom and stores it on a computer to propose an optimal prescription for a patient based on an input symptom of the patient and the adaptability (for example, see JP-A-2008-113807).

### BRIEF DESCRIPTION OF THE DRAWINGS

FIG. 1 is a block diagram illustrating an overall configuration of a prescription search system 1 to which the present invention is applied;

FIG. 2 is a block diagram of a search device constituting the prescription search system 1 to which the present invention is applied;

FIG. 3 is a drawing illustrating an example in which three levels or more of first degree of associations are preliminarily defined between each symptom and a prescription for a patient;

FIG. 4 is a drawing illustrating an example in which one or more prescriptions are associated with a combination of a plurality of symptoms via the first degree of associations;

FIG. 5 is a drawing illustrating a case where one or more prescriptions are associated with a combination of a symptom and a lifestyle habit and a combination of a symptom and a character via the first degree of associations;

FIG. 6 is a drawing illustrating a case where one or more prescriptions are associated with a combination of three kinds of a symptom, a lifestyle habit, and a character via the first degree of associations;

FIG. 7 is a drawing illustrating three or more levels of second degree of associations of prescriptions previously taken and respective improved symptoms of a patient who has taken the prescriptions with new prescriptions;

FIG. 8 is a drawing illustrating an example in which three or more levels of the first degree of associations are preliminarily defined between respective symptoms of a disease (lifestyle-related disease) and prescriptions for a patient;

FIG. 9 is a drawing illustrating an example in which one or more prescriptions are associated with a combination of a plurality of symptoms of the disease (lifestyle-related disease) via the first degree of associations;

FIG. 10 is a drawing illustrating three or more levels of the second degree of associations of prescriptions previously taken regarding the disease (lifestyle-related disease) and respective changed symptoms of a patient who has taken the prescriptions with new prescriptions;

FIG. 11 is a drawing illustrating an example in which three or more levels of the first degree of associations are preliminarily defined between respective symptoms of thin hair and prescriptions for a patient;

FIG. 12 is a drawing illustrating an example in which one or more prescriptions are associated with a combination of a plurality of symptoms of thin hair via the first degree of associations;

FIG. 13 is a drawing illustrating three or more levels of the second degree of associations of prescriptions previously taken for thin hair and respective changed symptoms of a patient who has taken the prescriptions with new prescriptions;

FIG. 14 is a drawing illustrating an example in which three levels or more of first degree of associations are preliminarily defined between respective symptoms regarding a beauty care and prescriptions for a patient;

FIG. 15 is a drawing illustrating an example in which one or more prescriptions are associated with a combination of a plurality of symptoms regarding the beauty care via the first degree of associations; and

FIG. 16 is a drawing illustrating three or more levels of the second degree of associations of prescriptions previously taken regarding the beauty care and respective changed symptoms of a patient who has taken the prescriptions with new prescriptions.

### DETAILED DESCRIPTION

However, to improve such a physical constitution and lifestyle habits, it is necessary to persistently select the optimal prescription suitable for the symptom and the lifestyle habits up to the present of the patient. Since the selection of the optimal prescription requires professional knowledge, advice from an expert having the knowledge is necessary. This has arisen a problem that, each time that the optimal prescription suitable for the symptom of the patient is selected, a time and a cost are required to obtain the advice from the expert.

Obviously, since the knowledge and a skill probably differ depending on the expert, the prescriptions proposed by the respective experts for the identical symptom often differ from one another. The difference in advice among the experts confuses the patient in some cases; therefore, the prescriptions proposed to the patient have needed to be uniformed. Further, there has been no effective reform measures to uniform the prescriptions thus proposed, and this has caused a problem of decrease in speed of improvement and accuracy.

Therefore, the present invention is invented in consideration of the above-described problems, and an object of which is to provide a non-transitory computer-readable recording medium storing allergy prescription search program that ensure automatically searching an optimal prescription from a symptom of a patient with allergy, such as pollinosis, and the like without an expert. Additionally, the object is to provide the non-transitory computer-readable recording medium storing prescription search program that ensure proposing optimal prescriptions for combinations of respective kinds of ingesta and respective pieces of attribute information with respective symptoms and ensure searching optimal prescriptions for respective symptoms affecting various symptoms, especially thin hair, and a beauty care.

An allergy prescription search system according to the present invention includes a database, input means, and search means. The database preliminarily stores three or more levels of first degree of associations between each symptom of allergy and a prescription for the symptom. With the input means, an actual symptom of allergy is input. The search means is configured to refer to the first degree of associations stored in the database to search one or more prescriptions based on the symptom input via the input means. The database preliminarily stores three or more levels of second degree of associations of a previously taken prescription found by the search by the search means and each improved symptom of a patient who has taken the prescription with a new prescription. With the input means, an improved symptom of the patient who has actually taken the previously taken prescription is input. The search means is configured to refer to the second degree of associations stored in the database to search for one or more new prescriptions based on the improved symptom input via the input means and the previously taken prescription.

An allergy prescription search system according to the present invention includes a database, input means, and search means. The database preliminarily stores three or more levels of second degree of associations of a previously taken prescription and each improved symptom of a patient who has taken the prescription with a new prescription. With the input means, a prescription previously actually taken and an improved symptom of the patient who has actually taken the prescription are input. The search means is configured to refer to the second degree of associations stored in the database to search for one or more new prescriptions based on the previously taken prescription and the improved symptom input via the input means.

An allergy prescription search method according to the present invention includes: an input step of inputting an actual symptom of an allergy; a search step of referring to three or more levels of first degree of associations between each symptom of an allergy and a prescription for the symptom stored in a database to search for one or more prescriptions based on the symptom input in the input step; a second degree of association obtaining step of obtaining three or more levels of second degree of associations of a previously taken prescription found by the search in the search step and each improved symptom of a patient who has taken the prescription with a new prescription; an improved symptom input step of inputting an actual improved symptom of the patient who has taken the previously taken prescription; and a new prescription search step of referring to the second degree of associations obtained in the second degree of association obtaining step to search for one or more new prescriptions based on the improved symptom input in the improved symptom input step and the previously taken prescription. The respective steps are executed by a computer.

An allergy prescription search method according to the present invention includes: a second degree of association obtaining step of obtaining three or more levels of second degree of associations of a previously taken prescription and each improved symptom of a patient who has taken the prescription with a new prescription; an improved symptom input step of inputting a previously actually taken prescription and an improved symptom of the patient who has actually taken the prescription; and a new prescription search step of referring to the second degree of associations obtained in the second degree of association obtaining step to search for one or more new prescriptions based on the previously taken prescription and the improved symptom input via the improved symptom input step. The respective steps are executed by a computer.

A non-transitory computer-readable recording medium storing allergy prescription search program according to the present invention causes a computer to execute: an input step of inputting an actual symptom of an allergy; a search step of referring to three or more levels of first degree of associations between each symptom of an allergy and a prescription for the symptom stored in a database to search for one or more prescriptions based on the symptom input in the input step; a second degree of association obtaining step of obtaining three or more levels of second degree of associations of a previously taken prescription found by the search in the search step and each improved symptom of a patient who has taken the prescription with a new prescription; an improved symptom input step of inputting an improved symptom of the patient who has actually taken the previously taken prescription; and a new prescription search step of referring to the second degree of associations obtained in the second degree of association obtaining step to search for one or more new prescriptions based on the improved symptom input in the improved symptom input step and the previously taken prescription.

A non-transitory computer-readable recording medium storing allergy prescription search program according to the present invention causes a computer to execute: a second degree of association obtaining step of obtaining three or more levels of second degree of associations of a previously taken prescription and each improved symptom of a patient who has taken the prescription with a new prescription; an improved symptom input step of inputting a previously actually taken prescription and an improved symptom of the patient who has actually taken the prescription; and a new prescription search step of referring to the second degree of associations obtained in the second degree of association obtaining step to search for one or more new prescriptions based on the previously taken prescription and the improved symptom input in the improved symptom input step.

A prescription search system to which the present invention is applied includes a database, input means, and search means. The database preliminarily stores three or more levels of first degree of associations between a combination of each symptom and any one or more of each lifestyle habit and each piece of attribute information, and a prescription for the symptom. With the input means, information including the combination is input. The search means is configured to refer to the first degree of associations stored in the database to search one or more prescriptions based on the information input via the input means. The database preliminarily stores three or more levels of second degree of associations of a previously taken prescription found by the search by the search means and each changed symptom of a patient who has taken the prescription with a new prescription. With the input means, a changed symptom of the patient who has actually taken the previously taken prescription is input. The search means includes the search means configured to refer to the second degree of associations stored in the database to search for one or more new prescriptions based on the changed symptom input via the input means and the previously taken prescription.

A prescription search system to which the present invention is applied includes a database, input means, and search means. The database preliminarily stores three or more levels of second degree of associations of a previous lifestyle habit and each changed symptom changed from previously with a new prescription. With the input means, an actual previous lifestyle habit and an actual changed symptom of a patient who has made the lifestyle habit are input. The search means is configured to refer to the second degree of associations stored in the database to search for one or more new prescriptions based on the previous lifestyle habit and the changed symptom input via the input means.

A non-transitory computer-readable recording medium storing prescription search program to which the present invention is applied causes a computer to execute: a first degree of association obtaining step of preliminarily obtaining three or more levels of first degree of associations of a combination of each symptom and any one or more of each lifestyle habit and each piece of attribute information with a prescription for the symptom; an input step of inputting information including the combination; a search step of referring to the first degree of associations obtained by the first degree of association obtaining step to search for one or more prescriptions based on the information input in the input step; a second degree of association obtaining step of obtaining three or more levels of second degree of associations of a previously taken prescription found by the search in the search step and each changed symptom of a patient who has taken the prescription with a new prescription; a changed symptom input step of inputting an actual changed symptom of the patient who has taken the previously taken prescription; and a new prescription search step of referring to the second degree of associations obtained in the second degree of association obtaining step to search for one or more new prescriptions based on the changed symptom input in the changed symptom input step and the previously taken prescription.

A non-transitory computer-readable recording medium storing prescription search program to which the present invention is applied causes a computer to execute respective steps of: a second degree of association obtaining step of obtaining three or more levels of second degree of associations of a previous lifestyle habit and each changed symptom changed from previously with a new prescription; a changed symptom input step of inputting an actual previous lifestyle habit and an actual changed symptom of a patient who has made the lifestyle habit; and a new prescription search step of referring to the second degree of associations obtained in the second degree of association obtaining step to search for one or more new prescriptions based on the previous lifestyle habit and the changed symptom input in the changed symptom input step.

A prescription search method to which the present invention is applied includes: a first degree of association obtaining step of preliminarily obtaining three or more levels of first degree of associations of a combination of each symptom and any one or more of each lifestyle habit and each piece of attribute information with a prescription for the symptom; an input step of inputting information including the combination; a search step of referring to the first degree of associations obtained by the first degree of association obtaining step to search for one or more prescriptions based on the information input in the input step; a second degree of association obtaining step of obtaining three or more levels of second degree of associations of a previously taken prescription found by the search in the search step and each changed symptom of a patient who has taken the prescription with a new prescription; a changed symptom input step of inputting an actual changed symptom of the patient who has taken the previously taken prescription; and a new prescription search step of referring to the second degree of associations obtained in the second degree of association obtaining step to search for one or more new prescriptions based on the changed symptom input in the changed symptom input step and the previously taken prescription. The respective steps are executed by a computer.

A prescription search method to which the present invention is applied includes: a second degree of association obtaining step of obtaining three or more levels of second degree of associations of a previous lifestyle habit and each changed symptom changed from previously with a new prescription; a changed symptom input step of inputting an actual previous lifestyle habit and an actual changed symptom of a patient who has made the lifestyle habit; and a new prescription search step of referring to the second degree of associations obtained in the second degree of association obtaining step to search for one or more new prescriptions based on the previous lifestyle habit and the changed symptom input in the changed symptom input step. The respective steps are executed by a computer.

The present invention having the above-described configurations allows determining the prescription from the symptom newly obtained via the operating unit with reference to the first degree of associations.

Moreover, the present invention can automatically execute these determination operations without manpower. This eliminates the need for taking a labor cost and time required for staff having professional knowledge performing analysis on the newly obtained symptom.

The following describes an allergy prescription search system to which the present invention is applied in detail with reference to the drawings.

### First Embodiment

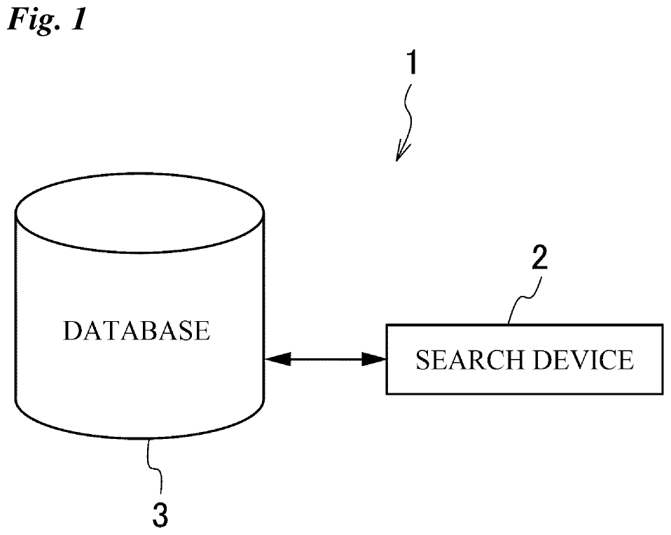

FIG. 1 is a block diagram illustrating an overall configuration of a prescription search system 1 to which the present invention is applied. The prescription search system 1 automatically searches an optimal prescription from a symptom of a patient with allergy, such as pollinosis, and the like, without an expert. Here, while the allergy includes all allergies, such as food and an environment, not only pollinosis, the following gives a description with pollinosis as an allergy from pollen, such as a Japanese cedar, as an example.

The prescription search system 1 includes a database 3 and a search device 2 coupled to the database 3. As the database 3, a database regarding prescriptions to be provided to patients is constructed. In the database 3, information transmitted via a public telecommunication network or information input by a user of the system is accumulated. Additionally, based on a request from the search device 2, the accumulated information is transmitted from the database 3 to the search device 2. The database 3 may be controlled by an artificial intelligence. The artificial intelligence may be based on any well-known artificial intelligence technology.

Although the search device 2 is configured of an electronic device, for example, a personal computer (PC) and the like, except for the PC, the search device 2 may be embodied with all other electronic devices, such as a mobile phone, a smart phone, a tablet terminal, and a wearable device.

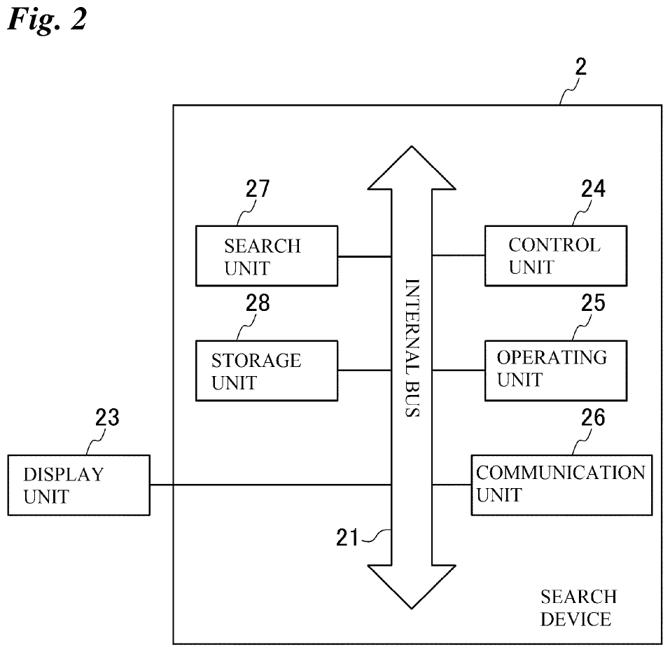

FIG. 2 illustrates an example of a specific configuration of the search device 2. In the search device 2, a control unit 24, which controls the entire search device 2, an operating unit 25, which inputs a command for various controls via an operating button, a keyboard, and the like, a communication unit 26 for wired communications or wireless communications, a search unit 27, which searches for optimal detection algorithm information, and a storage unit 28 represented by a hard disk and the like, which stores programs for searching to be executed, are each coupled to an internal bus 21. Furthermore, to the internal bus 21, a display unit 23 as a monitor to actually display information is coupled.

The control unit 24 is what is called a center control unit that transmits a control signal via the internal bus 21 to control the respective components mounted in the search device 2. The control unit 24 transmits commands for various controls according to the operations via the operating unit 25 via the internal bus 21.

The operating unit 25 is embodied with a keyboard and a touchscreen, and an execution instruction to execute the program is input by the user. When the execution instruction is input by the user, the operating unit 25 notifies the control unit 24 of it. The control unit 24 that has received the notification executes a desired process operation by causing each component, such as the search unit 27, to collaborate.

Via the operating unit 25, a symptom, ingesta, and a lifestyle habit of a patient who is actually prescribed and attribute information and a character of the patient are further input. When the user himself/herself is the patient, the patient himself/herself inputs these pieces of information via the operating unit 25. When the user is a consultant giving advice to the patient, the consultant inputs the information heard from the patient via the operating unit 25.

The search unit 27 searches an optimal prescription for the patient from the information input via the operating unit 25. To execute the search operation, the search unit 27 reads various pieces of information stored in the storage unit 28 as required information. The search unit 27 may be controlled by an artificial intelligence. The artificial intelligence may be based on any well-known artificial intelligence technology.

The display unit 23 is configured of a graphic controller that produces a display image based on the control by the control unit 24. The display unit 23 is achieved with, for example, a liquid crystal display (LCD).

In a case where the storage unit 28 is constituted of a hard disk, predetermined information is written in each address based on the control by the control unit 24 and is read as necessary. The storage unit 28 stores the program to execute the present invention. The program is read and executed by the control unit 24.

Next, the following describes an operation by the prescription search system 1 having the above-described configuration.

First, the symptom, the ingesta, the lifestyle habit, the attribute, and the character of the patient who is actually prescribed are input via the operating unit 25. The input information is transmitted to the search unit 27 and the database 3.

The search unit 27 searches for an optimal prescription for the patient based on the information transmitted from the operating unit 25. In a process of searching, the search unit 27 refers to the information stored in the database 3.

The database 3 preliminarily stores three or more levels of first degree of associations of respective symptoms, respective kinds of ingesta, respective lifestyle habits, respective pieces of attribute information, and respective characters of the patient with the prescription for the patient. The first degree of association only needs to be at least associated between at least each symptom and the prescription for the patient, and associations with each kind of the ingesta, each lifestyle habit, each piece of the attribute information, and each character of the patient via the first degree of association are not especially required.

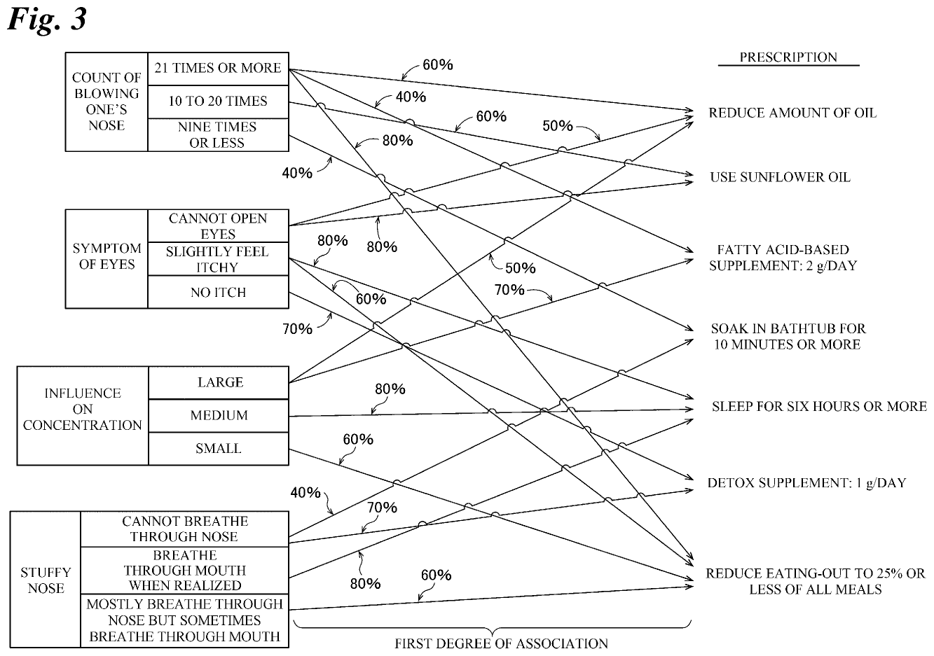

FIG. 3 illustrates an example in which three levels or more of the first degree of associations are preliminarily defined between each symptom and the prescription for the patient. The respective symptoms are arrayed on the left side via the first degree of associations, and the respective prescriptions are arrayed on the right side via the first degree of associations. The first degree of association indicates a degree that which prescription has a high relevance to the symptom arrayed on the left side. In other words, the degree of association is an index indicating that each symptom is the most likely to be associated with which prescription and indicating appropriateness to select an optimal prescription from the symptom.

The symptom is an item indicative of the actual symptom of pollinosis of the patient. Examples of the items of the symptom include “count of blowing one's nose” (21 times or more, 10 to 20 times, nine times or less), “symptom of eyes” (cannot open eyes, slightly feel itchy, no itch), “influence on concentration” (large, medium, small), “stuffy nose” (cannot breathe through nose, breathe through mouth when realized, mostly breathe through nose but sometimes breathe through mouth), and the like.

The ingesta include any kind of ingesta ingestible by the patient, such as food, a beverage, a supplement, and a medical agent.

The attribute information of the patient includes information, such as an age, a sex, an occupation, presence/absence of a housemate, and the like, whether a relative affected with pollinosis exists within the second degree, or results of a medical examination by medical institution.

Table 1 summarizes examples of these symptoms. Note that the symptoms are not limited to the examples in Table 1, and as long as similar to these symptoms, any other item is included.

TABLE 1

| | Item (Large | | |
|---|---|---|---|
| | Classification) | Item (Small Classification) | Details |
| Symptom | Symptom of | Symptom of eyes | Itch and pain to the extent of not being able to open eyes, feel itchy but can go |
| | pollinosis in | | out somehow, slightly feel itchy, no itch |
| | this year | Necessity for mask | Entirely impossible to go out, can go out somehow with tissue or the like, |
| | | | slightly feel discomfort but can go out without significant problem, can live with |
| | | | no problem |
| | | Influence on concentration | Symptom hardly keeps concentration, cannot produce result as usual but still |
| | | | can concentrate, slightly feel discomfort but does not mind pollinosis while |
| | | | concentrating, can concentrate with no problem |
| | | Influence on meal | Have no appetite, can eat but cannot enjoy meal in many cases, slightly feel |
| | | | discomfort but can eat smoothly, can eat with no problem |
| | | Influence on sleep | Awake many times and do not feel asleep, feel asleep but insufficient, |
| | | | sometimes awake but have satisfaction of sleep, can sleep with no problem |
| | | Count of blowing one's nose | Detrimental to daily life (yardstick: 21 times or more), inconvenient but has no |
| | | | problem in daily life (yardstick: 11 to 20 times), slightly inconvenient but has no |
| | | | significant problem (yardstick: 10 times), has no problem (yardstick: nine times |
| | | | or less) |
| | | Count of sternutation | Detrimental to daily life (yardstick: 21 times or more), inconvenient but has no |
| | | | problem in daily life (yardstick: 11 to 20 times), slightly inconvenient but has no |
| | | | significant problem, (yardstick: 10 times), has no problem (yardstick: nine |
| | | | times or less) |
| | | Situation of stuffy nose | Cannot breathe through nose patently, often breathe through mouth when |
| | | | realized, mostly breathe through nose but sometimes breathe through mouth, |
| | | | no stuffy nose |
| Ingesta | Usual meal | Tendency of amount of oil | Large, neither of them, small |
| | in this year | Tendency of kinds of oil | Many, neither of them, few |
| | | Tendency of kinds of meals | Many, neither of them, few |
| | | Percentage of eating-out | Eating-out by 75% or more, eating-out by 50 to 75%, eating-out by 25 to |
| | | | 50%, eating-out by 255 or less |
| | | Situation of ingression of | Substantially more than 2 L/day, about 2 L/day, substantially less than 2 L/day |
| | | water | |
| | Usage situation | Usage | Used, not used |
| | of supplements | (When used) | Period of use · frequency |
| | in this year | | |
| | Situation of | Usage | Used, not used |
| | use of various | (When used) | XXXX (Name of medicine) |
| | medicines/various | | |
| | cures in this | | |
| | year | | |
| | Situation of | Usage | Used, not used |
| | use of medicine | (When used) | Period of use · frequency |
| | other than | | |
| | medicine for | | |
| | pollinosis | | |
| | in this year | | |
| Attribute | Basic attribute | Sex | Male, female |
| information of | | Birthdate | Jun. 30, 1969 |
| patient | | Occupation (Work at night | Day duty, night duty, irregular between day duty and night duty |
| | | or not) | |
| | | Married/unmarried | Married, unmarried |
| | | Housemate | Have housemate who cooks, no housemate who cooks |
| | Pollinosis | Presence of relative within | Yes, No |
| | heredity | second degree affected with | |
| | | pollinosis | |
| | | Presence of co-resident | Yes, No |
| | | family affected with pollinosis | |
| | History of | Onset of disease | Yes, No |
| | pollinosis | (When yes) | XX years have passed after onset of disease |
| | Minerals in hair | Contents of various minerals | Calcium: 118: 350 μg/kg, zinc 21, 152 μg/Kg, and the like |

The prescription includes items regarding a meal and a lifestyle habit to improve pollinosis, such as “reduce amount of oil,” “use sunflower oil,” “fatty acid-based supplement: 2 g/day,” “soak in bathtub for 10 minutes or more,” “sleep for six hours or more,” “detox supplement: 1 g/day,” and “reduce eating-out to 25% or less of all meals.”

Table 2 summarizes examples of these prescriptions. However, the prescriptions are not limited to the examples in Table 2 and as long as similar to these prescriptions, any other item is included.

TABLE 2

| | | |
|---|---|---|
| Meal | Amount of oil | Reduce amount of use of oil |
| | | Maintain amount of use of oil |
| | Kind of oil | Change oil usually used to olive oil |
| | | Change oil usually used to rapeseed oil or sunflower oil |
| | Tendency of | Stop eating egg dishes and increase fish dishes |
| | kind of meal | Reduce eating-out to 25% or less of all meals |
| Supplement | Kind to be | Fatty acid-based supplement: 2 g/day |
| | ingested 1 | Fatty acid-based supplement: 1 g/day |
| | Kind to be | Detox supplement: 2 g/day |
| | ingested 2 | Detox supplement: 1 g/day |
| Rehydration | Amount of | Ingest water by 3 L or more/day |
| | ingested water | Ingest water by 2 L or more/day |
| Exercise | Amount of | Exercise for 20 minutes or more: two days or more/week |
| | exercise | Exercise for 20 minutes or more: five days or more/week |
| Bath | Amount of bath | Bathe (soak in bathtub) for 10 minutes or more: two days or more/week |
| | | Bathe (soak in bathtub) for 10 minutes or more: five days or more/week |
| Sleep | Amount of sleep | Have deep sleep for six hours or more: three days or more/week |
| | | Have deep sleep for six hours or more: five days or more/week |

While the prescription is roughly classified into the meal, the supplement, the rehydration, the exercise, the bath, the sleeping, and the like, the prescriptions are not limited to these. As long as a prescription is a factor contributing to improve pollinosis, any prescription is included.

For example, when “count of blowing one's nose” is 21 times or more, it is indicated that the first degree of association of “reduce amount of oil” is 60%, the first degree of association of “fatty acid-based supplement: 2 g” is 40%, and the first degree of association of “reduce eating-out to 25% or less of all meals” is 80%. When “count of blowing one's nose” is 10 to 20 times, the first degree of association of “use sunflower oil” is indicated as 60%. When “count of blowing one's nose” is nine times or less, the first degree of association of “soak in bathtub for 10 minutes or more” is 40%.

When “influence on concentration” is large, the first degree of association of “reduce amount of oil” is 50%, and the first degree of association of “fatty acid-based supplement: 2 g” is 70%. When “influence on concentration” is medium, the first degree of association of “sleep for six hours or more” is 80%. When “influence on concentration” is small, the first degree of association of “reduce eating-out to 25% or less of all meals” is 60%.

The first degree of association may be constituted by a model that can be updated through what is called machine learning and may be constituted by a neural network. Alternatively, the first degree of association may be constituted by a network on the premise of deep learning.

The search unit 27 thus refers to the first degree of association stored in the database 3 and determines that each symptom newly input via the operating unit 25 corresponds to which symptom arrayed on the left side of the first degree of association. Assume that when the symptom newly input via the operating unit 25 is “slightly feel itchy” in “symptom of eyes,” the search can find that “sleep for six hours or more” with the first degree of association of 80% is the most suitable prescription, and the search can find that “reduce eating-out to 25% or less of all meals” with the first degree of association of 60% is a prescription as a second opinion.

The search unit 27 refers to these first degree of associations and executes a work to select the prescription based on the symptom newly obtained via the operating unit 25. At this time, the search unit 27 may select the prescription with the highest first degree of association. Because, as described above, the higher the first degree of association is, appropriateness of the selection becomes high. However, the search unit 27 is not limited to the case where the prescription with the highest first degree of association is selected, a prescription with the medium first degree of association or a prescription with the low first degree of association may be purposely selected. Except for these, it is obvious that a prescription with the first degree of association of 0% where a symptom and a prescription are not connected with an arrow may be selected. The search unit 27 is not limited to the case of selecting one prescription but may purposely select a plurality of prescriptions with reference to the first degree of associations. The prescription searched by the search unit 27 is displayed via the display unit 23.

Note that the respective items of the symptoms and the prescriptions included in FIG. 3 are one example, and it is obvious that the process operation is executed similarly to the other items exemplified in Table 1 and Table 2 described above.

That is, the prescription search system 1 to which the present invention is applied allows determining the prescription from the symptom newly obtained via the operating unit 25 with reference to the above-described first degree of associations. Moreover, the prescription search system 1 to which the present invention is applied can automatically execute these determination operations without manpower. This eliminates the need for taking a labor cost and time required for staff having professional knowledge performing analysis on the newly obtained symptom.

The prescription search system 1 to which the present invention is applied has a feature in searching the prescription via the first degree of associations set to three or more levels. While the first degree of association can be described by a value, for example, from 0 to 100%, the first degree of association is not limited to this. As long as the first degree of association can be described by three or more levels of values, the first degree of association may be configured into any level.

By the search based on the first degree of associations expressed by three or more levels of values, executing the search and display in an order of the high degree of associations under a condition where a plurality of prescriptions are selected is possible. When thus the first degree of associations can be displayed in the order of the high first degree of associations for the user, a prescription with a higher possibility can be preferentially selected and displayed. Meanwhile, even the prescription with the low first degree of association, a prescription specified from that can be displayed in the meaning of a second opinion, thereby ensuring providing usefulness in a case where a symptom is not improved by a prescription displayed as a first pinion or a similar case.

In addition, the present invention allows determination without missing a prescription with the extremely low first degree of association, such as 1%. This allows calling user's attention to a fact that even the prescription with the extremely low first degree of association, the prescription is related as a slight sign and therefore there may be a case where the prescription is useful as a beneficial prescription once in several ten times or several hundred times.

Further, according to the present invention, the search based on the three or more levels of the first degree of associations is advantageous in that search policy can be determined by way of setting a threshold. While decreasing the threshold allows picking up even the prescription with the above-described first degree of association of 1% without missing, there is a possibility that many prescriptions based on the first degree of associations having a low possibility of correct solution are picked up. While increasing the threshold allows narrowing down prescriptions only to prescriptions having a high possibility of correct solution, there may be a case where a prescription suggesting a preferred solution is missed once in several ten times or several hundred times. While which one is regarded as important can be determined based on a way of thinking by a user side or a system side, a degree of freedom for selection of the point regarded as important can be increased.

Further, with the present invention, the above-described first degree of associations may be updated. That is, the symptoms and the prescriptions as depicted in FIG. 3 are updated as needed. This update may be executed by reflecting the information provided via a public telecommunication network, for example, the Internet. Alternatively, the system side or the user side may artificially or automatically update the first degree of associations based on contents of, for example, research data and a thesis by an expert, a presentation at a conference, a newspaper article, and a book. An artificial intelligence may be used for these update processes.

The first degree of association is updated by increasing or decreasing the first degree of association whenever information regarding a relation between a symptom and a prescription for the symptom is input. For example, when it can be newly confirmed that a certain prescription is effective to a certain symptom through a thesis, a presentation at a conference, other research data based on experimental verification, and the like, the degree of association between the symptom and the prescription is increased. Alternatively, when it can be newly confirmed that a certain prescription is not effective to a certain symptom through a thesis, a presentation at a conference, other research data based on experimental verification, and the like, the first degree of association between the symptom and the prescription is decreased.

The first degree of associations are set to the three or more levels as described above, and this allows freely handling the first degree of association when the first degree of association is desired to increase and decrease. The update of the first degree of association itself may be executed through the above-described machine learning and deep learning.

Further, in a case where a new symptom that has not existed up to the present has been found or in a case where a new prescription that has not existed up to the present has been found, a new first degree of association may be set between them. The first degree of association of these new symptom and prescription may be updated as described above.

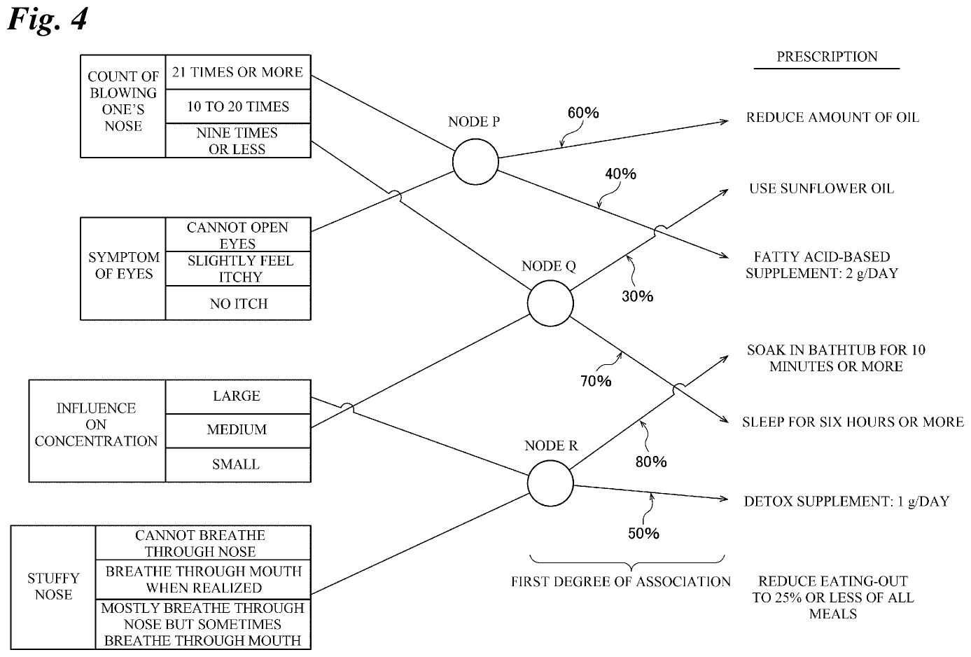

The above-described first degree of association is not limited to the case where a prescription is associated with a single symptom. For example, as illustrated in FIG. 4, one or more prescriptions may be associated with a combination of a plurality of symptoms via the first degree of associations.

In the example of FIG. 4, a node P, which is a combination of “21 times or more” of “blow one's nose” and “cannot open eyes” of “symptom of eyes,” a node Q, which is a combination of nine times or less of “blow one's nose” and “medium” of “influence on concentration,” and a node R, which is a combination of “large” of “influence on concentration” and “breathe through mouth when realized” of “stuffy nose,” are each provided. In the node P, the first degree of association of “reduce amount of oil” is 60%, and the first degree of association of “fatty acid-based supplement: 2 g/day” is 40%. In the node Q, the first degree of association of “use sunflower oil” is 30%, and the first degree of association of “sleep for six hours or more” is 70%. In the node R, the first degree of association of “soak in bathtub for 10 minutes or more” is 80%, and the first degree of association of “detox supplement: 1 g/day” is 50%.

The first degree of associations of these combinations are preliminarily obtained. Next, the search unit 27 refers to the first degree of associations and determines whether two or more symptoms newly input via the operating unit 25 each correspond to which symptom arrayed on the left side of the first degree of association. Assume that when the symptoms newly input via the operating unit 25 are “21 times or more” of “count of blowing one's nose” and to the extent of “cannot open eyes” of “symptom of eyes,” this corresponds to the node P. Therefore, “reduce amount of oil” with the first degree of association of 60% or “fatty acid-based supplement: 2 g/day” with the first degree of association of 40% in the node P or the like is selected.

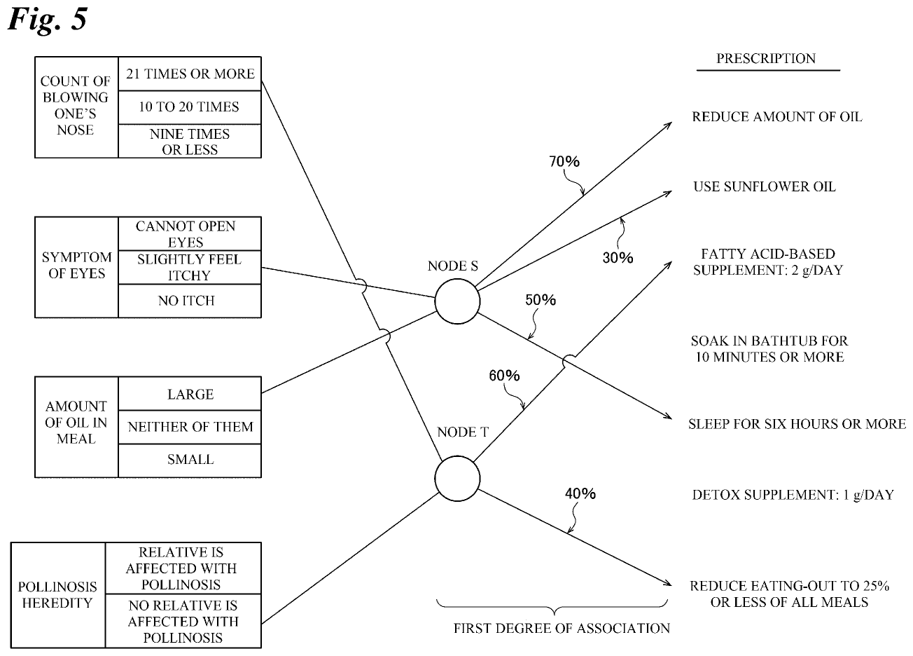

An example of FIG. 5 illustrates a case where one or more prescriptions are associated with a combination of the symptom and the ingesta and a combination of the symptom and the attribute information of the patient via the first degree of associations.

A node S, which is a combination of “slightly feel itchy” of “symptom of eyes” and “large” of “amount of oil in meal” as the ingesta, and a node T, which is a combination of “21 times or more” of “blow one's nose” and “no relative is affected with pollinosis” of the attribute information of the patient, are each provided. In the node S, the first degree of association of “reduce amount of oil” is 70%, the first degree of association of “sleep for six hours or more” is 50%, and the first degree of association of “use sunflower oil” is 30% for each prescription. In the node T, the first degree of association of “fatty acid-based supplement: 2 g/day” is 60%, and the first degree of association of “reduce eating-out to 25% or less of all meals” is 40% for each prescription.

The first degree of associations of these combinations are preliminarily obtained. Next, the search unit 27 refers to the first degree of associations and determines whether a combination of a symptom and ingesta and a combination of a symptom and attribute information newly input via the operating unit 25 correspond to which item arrayed on the left side of the first degree of association. Assume that when the symptom is “slightly feel itchy” of “symptom of eyes” and the ingesta are “large” of “amount of oil in meal,” which are newly input via the operating unit 25, these correspond to the node S, and respective items associated with the node S by the first degree of associations are selected. Similarly, when the symptom is “21 times or more” of “count of blowing one's nose” and the attribute information of the patient is “no relative is affected with pollinosis,” which are newly input via the operating unit 25, these correspond to the node T, and respective items associated with the node T by the first degree of associations are selected.

In view of this, in addition to the symptoms, preliminarily specifying the first degree of associations based on the ingesta and the attribute information allows further highly accurate search.

Note that the respective items of the symptoms, the ingesta, the attribute information, and the prescriptions included in FIG. 5 are one example, and it is obvious that the process operation is executed similarly to the other items exemplified in Table 1 and Table 2 described above.

With the above-described first degree of associations of the combinations illustrated in FIG. 5, a combination of one symptom and two or more kind of ingesta, a combination of two or more symptoms and one kind of ingesta, and a combination of two or more symptoms and two or more kind of ingesta may be associated. Similarly, with the above-described first degree of associations, a combination of one symptom and two or more pieces of attribute information, a combination of two or more symptoms and one piece of attribute information, and a combination of two or more symptoms and two or more pieces of attribute information may be associated.

From an aspect of improving a convenience of input of the symptom, the ingesta, and the attribute information via the operating unit 25, a configuration in which various questions are displayed in the display unit 23 and the user answers the question by operating the operating unit 25 such that the user can spontaneously input these pieces of information may be employed.

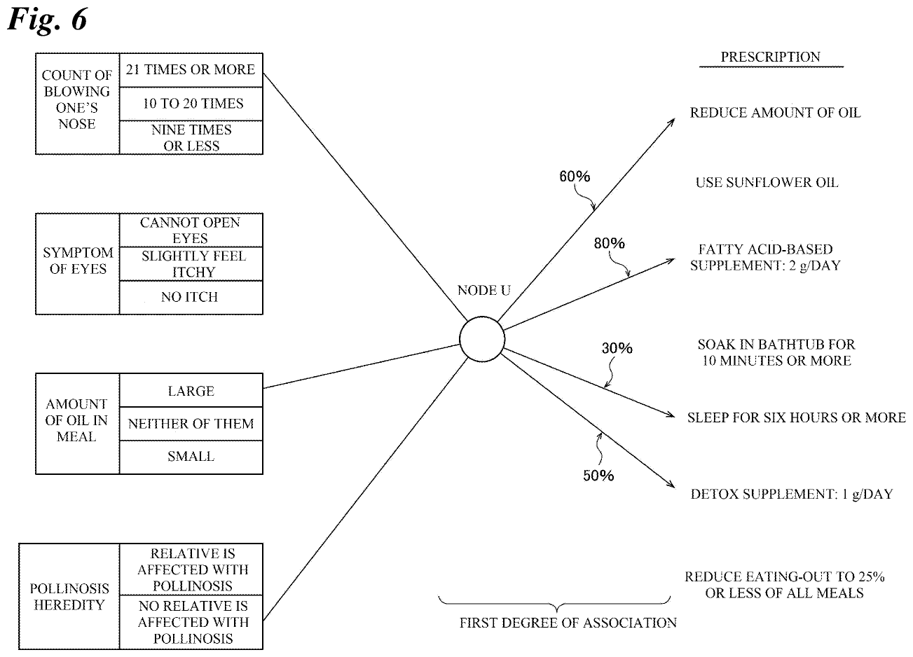

The example of FIG. 6 illustrates the case where one or more prescriptions are associated with a combination of three kinds of a symptom, ingesta, and attribute information via the first degree of associations.

A node U, which is a combination of “21 times or more” of “count of blowing one's nose,” “large” of “amount of oil in meal” as the ingesta, and “no relative is affected with pollinosis” of the attribute information, is provided. In the node U, the first degree of association of “reduce amount of oil” is 60%, the first degree of association of “fatty acid-based supplement: 2 g/day” is 80%, the first degree of association of “sleep for six hours or more” is 30%, and the degree of association of “detox supplement: 1 g/day” is 50% for each prescription.

The first degree of associations of these combinations are preliminarily obtained. Next, the search unit 27 refers to the first degree of associations and determines whether each combination of the symptom, the ingesta, and the attribute information newly input via the operating unit 25 corresponds to which item arrayed on the left side of the first degree of association. Assume that when the symptom is “21 times or more” of “count of blowing one's nose,” “amount of oil in meal” as the ingesta is “large,” and the attribute information is “no relative is affected with pollinosis,” which are newly input via the operating unit 25, these correspond to the node U, and respective items associated with the node U with the first degree of associations are selected. In view of this, preliminarily specifying the first degree of associations based on the ingesta and the attribute information in addition to the symptoms allows further highly accurate search.

Note that the respective items of the symptoms, the ingesta, the attribute information, and the prescriptions included in FIG. 6 are one example, and it is obvious that the process operation is executed similarly to the other items exemplified in Table 1 and Table 2 described above.

As long as a combination is one or more symptoms, one or more kind of ingesta, and one or more pieces of attribute information, any combination may be used with the first degree of associations for the combinations illustrated in FIG. 6 described above. The respective first degree of associations illustrated in FIGS. 5 and 6 may be similarly updatable.

With the present invention, as the items arrayed on the left side of the first degree of associations, in addition to the symptom, the ingesta, and the attribute information, external information, and personal information and the character of the patient may be added.

The external information includes, for example, information on an environment, such as data of an amount of scattered pollen in a current residence, weather, and a temperature.

The lifestyle habit includes information on exercise, bath, sleeping, a situation of going out, a residence, and the like.

The following Table 3 shows an example of the lifestyle habit, the external information, and the character of the patient. Note that these lifestyle habit, external information, and character of the patient are not limited to Table 3, and any other lifestyle habit, external information, and character of the patient are included.

TABLE 3

| | Item (Large Classification) | Item (Small Classification) | Details |
|---|---|---|---|
| Lifestyle Habit | Exercise in this year | Execution situation of | Exercise for 20 minutes or more: two days or more/week, exercise |
| | | exercise | for 20 minutes or more: about one day/week, hardly exercise for 20 |
| | | | minutes or more |
| | Bath in this year | Situation of bath | Bathe (soak in bathtub) for 10 minutes or more: two days or |
| | | | more/week, bathe (soak in bathtub) for 10 minutes or more about |
| | | | one day/week, hardly bathe (soak in bathtub) for 10 minutes or |
| | | | more |
| | Sleep in this year | Situation of sleep | Have deep sleep for six hours or more, cannot have deep sleep for |
| | | | six hours or more |
| | Situation of going out in this | Places visited | Urban center, suburbs (flat area), suburbs (mountains and forests) |
| | year | Frequency of going out | Six times or more per week, four or five times per week, three |
| | | | times or less per week |
| | Main residence in this year | XX prefecture | Tokyo, Aichi prefecture, Hyogo prefecture |
| | Going to hospital due to | Whether going to hospital | Going to hospital, not going to hospital |
| | pollinosis in this year | | |
| Character | Improvability of character | Character of user | Can spend regular life, neither of them, not good at spending regular |
| | | | life |
| External | Amount of scattered pollen | XXX | +150% compared with criterion |
| Information | in residence in last year | | |
| | Amount of scattered pollen | XXX | −20% compared with criterion |
| | in residence in last year | | |

By associating the relation between the combination of the lifestyle habit, the external information, and the character and the symptom of the patient and the prescription via the first degree of association, further highly accurate search considering the lifestyle habit, the external information, and the character of the patient can be executed with reference to these first degree of associations. At this time, obviously, in addition to the combination of any one or more of the lifestyle habit, the external information, and the character of the patient with the symptom, a relation between a combination of the above-described ingesta and/or attribute information and the prescription may be further included in the first degree of association.

### Second Embodiment

The following describes the prescription search system 1 according to the second embodiment to which the present invention is applied. In this second embodiment, the identical reference numerals are given to components and members identical to those of the above-described first embodiment, and therefore the following omits the descriptions.

This second embodiment refers to the database 3 in which prescriptions previously taken, respective improved symptoms of the patient who has performed the prescriptions, and three or more levels of second degree of associations with new prescriptions are preliminarily stored.

Here, the previously taken prescription includes any previously taken prescription for the patient, regardless of whether the prescription is the prescription searched through the prescription search system 1 to which the present invention is applied. As the prescriptions previously taken, the examples shown in Table 2 and the like are considered; however, the prescriptions are not limited to these. As long as the item relates to the meal and the lifestyle habit to improve pollinosis, any other item is included. As other prescriptions previously taken, a name of a prescription medicine, a period of hyposensitization therapy, a name of an ingested supplement, a frequency of ingestion, and the like are included.

Additionally, although the improved symptom of the patient who has taken the prescription is similar to the example of the symptoms shown in Table 1 as the example of the content of the item, the improved symptom is not limited to these. As long as an item indicates the symptom of pollinosis, any other item is included.

As the new prescription, although the examples shown in Table 2 and the like are considered, the prescription is not limited to these. As long as an item relates to the meal and the lifestyle habit to improve pollinosis, any other item is included.

Next, the following describes an operation by the prescription search system 1 according to the second embodiment. First, the previously taken prescription and each improved symptom of the patient who has taken the prescription are input via the operating unit 25. The input information is transmitted to the search unit 27 and the database 3.

The search unit 27 searches for a new prescription for the patient based on the information transmitted from the operating unit 25. In a process of searching, the search unit 27 refers to the information stored in the database 3.

The database 3 preliminarily stores the above-described second degree of associations. The second degree of association indicates a degree of which new prescription is highly related to a combination of each previously taken prescription and each improved symptom arrayed on the left side. In other words, the second degree of association is an index indicating that a combination of each symptom previously taken and each improved symptom is the most likely to be associated with which new prescription and indicating appropriateness to select an optimal prescription from the symptom.

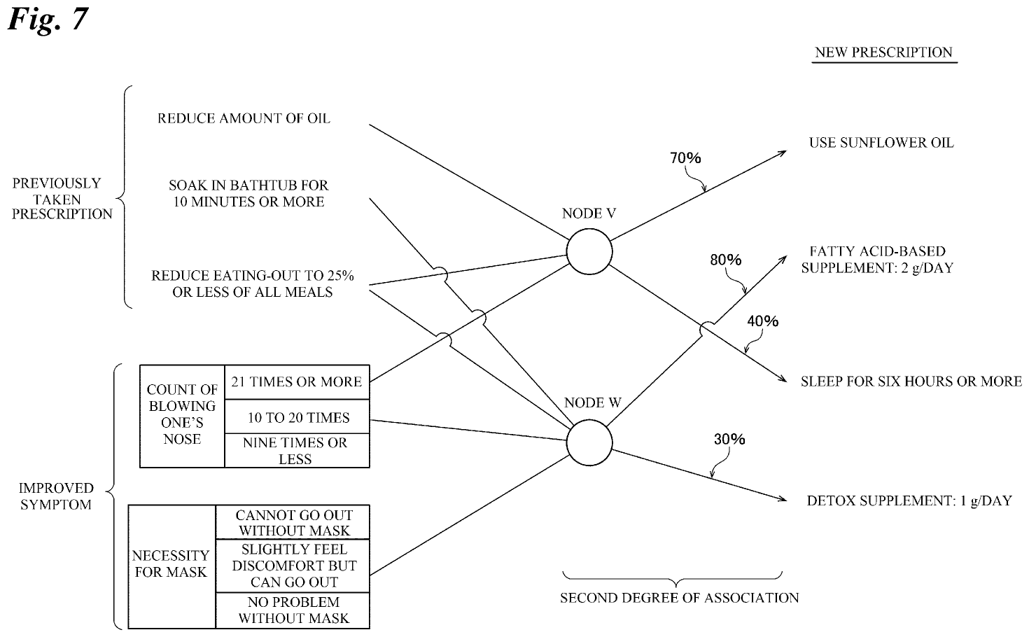

FIG. 7 illustrates an example of the second degree of associations. The respective prescriptions previously taken and the respective improved symptoms are arrayed on the left side via the second degree of associations, and the respective new prescriptions are arrayed on the right side via the second degree of associations.

In the example of FIG. 7, a node V, which is a combination of “reduce amount of oil” and “reduce eating-out to 25% or less of all meals” as the prescriptions previously taken and “21 times or more” of “count of sternutation” per unit time as the improved symptom, and a node W, which is a combination of “soak in bathtub for 10 minutes or more” and “reduce eating-out to 25% or less of all meals” as the prescriptions previously taken and “10 to 20 times” of “count of sternutation” per unit time and “slightly feel discomfort but can go out” of “necessity for mask” as the improved symptoms, are each provided. In the node V, the second degree of association of the new prescription, “use sunflower oil” is 70%, and the second degree of association of “sleep for six hours or more” is 40%. Additionally, in the node W, the second degree of association of the new prescription, “fatty acid-based supplement: 2 g/day” is 80%, and the second degree of association of “detox supplement: 1 g/day” is 30%.

The second degree of associations of these combinations are preliminarily obtained. Next, the search unit 27 refers to the second degree of associations and determines whether the previously taken prescription and each improved symptom of the patient who has taken the prescription, which are newly input via the operating unit 25, each correspond to which item arrayed on the left side of the second degree of associations. Assume that when the prescriptions previously taken input via the operating unit 25 are “soak in bathtub for 10 minutes or more” and “reduce eating-out to 25% or less of all meals” and the improved symptoms input via the operating unit 25 are “10 to 20 times” of “count of sternutation” and “slightly feel discomfort but can go out” of “necessity for mask,” these correspond to the node W. In this case, with reference to the second degree of associations of the node W, “fatty acid-based supplement: 2 g/day,” “detox supplement: 1 g/day,” and the like are selected as the new prescriptions.

Thus, in the second embodiment, a new prescription can be automatically searched based on the previously taken prescription and each improved symptom of the patient who has taken the prescription. In view of this, executing the second embodiment continuously from the first embodiment allows continuously observing the symptom of pollinosis of the patient and selecting an optimal prescription according to the symptom. In a case where the previously taken prescription is comparatively strong and the symptom of the patient is improved after that, the best prescription can be continuously proposed timely, such as switching the prescription to a prescription less effective compared with the previous prescription.

Incidentally, the second degree of association may be similarly updated. That is, the previously taken prescription, each improved symptom, and the new prescription as depicted in FIG. 7 are updated as needed. This update may be executed by reflecting the information provided via a public telecommunication network, for example, the Internet. Alternatively, the system side or the user side may artificially or automatically update the second degree of associations based on contents of, for example, research data and a thesis by an expert, a presentation at a conference, a newspaper article, and a book. An artificial intelligence may be used for these update processes.

Obviously, the second degree of association may associate between a combination of one or more of any of the above-described ingesta, attribute information, lifestyle habit, character, and external information, in addition to the previously taken prescription and each improved symptom, and the prescription.

### Third Embodiment

The following describes the prescription search system 1 according to the third embodiment to which the present invention is applied. In this third embodiment, the identical reference numerals are given to components and members identical to those of the above-described first embodiment and second embodiment, and therefore the following omits the descriptions.

### Search for Prescription for Disease

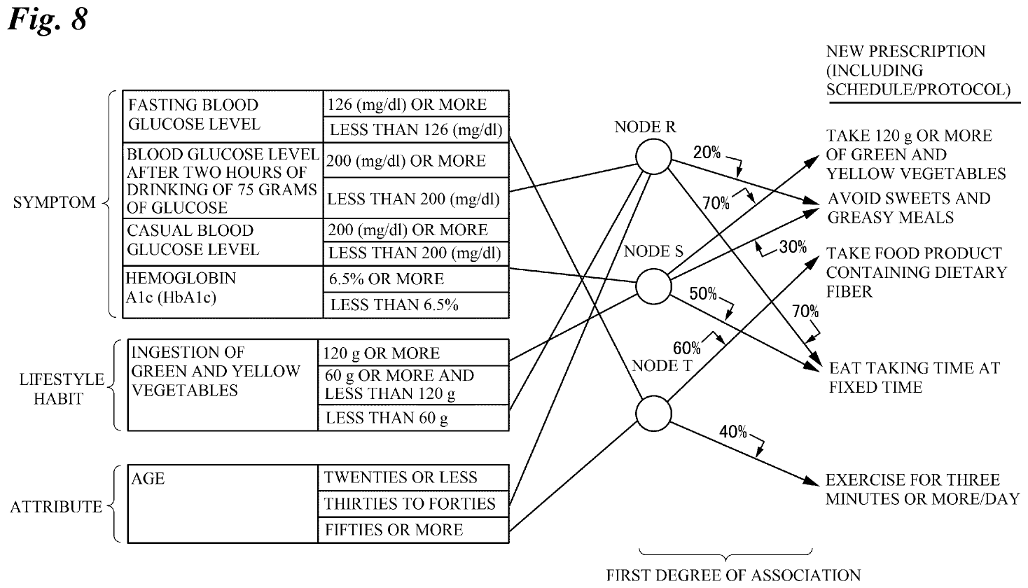

FIG. 8 illustrates an example in which three or more levels of the first degree of associations are preliminarily defined between respective symptoms of a disease and prescriptions for the patient. The respective symptoms are arrayed on the left side via the first degree of associations, and the respective prescriptions are arrayed on the right side via the first degree of associations. The first degree of association indicates a degree that which prescription has a high relevance to the symptom arrayed on the left side. In other words, the degree of association is an index indicating that each symptom is the most likely to be associated with which prescription and indicating appropriateness to select an optimal prescription from the symptom.

In addition to these, the first degree of association associates one or more prescriptions with a combination of each symptom, ingesta, and attribute information.

The symptoms are various symptoms of a disease and include, for example, a direct symptom, such as a blood glucose level and an amount of hemoglobin A1c, and an indirect symptom, such as various test values other than these in medical examination and the amount of minerals inside of a body. These symptoms include indexes expressed in various medical treatment data, results (including a VAS evaluation) sensed by a doctor, the patient, an estimator, and the like as sensed values, and the like.

The lifestyle habit includes all events related to the life of the patient. Examples of the lifestyle habit regarding an eating habit include the ingesta and an amount of a meal ingested during the meal, time of the meal, and the like. The ingesta here include any kind of ingesta ingestible by the patient, such as food, a beverage, a supplement, and a medical agent. The lifestyle habits regarding a sleep include hours of sleep, hour of rising, bedtime, and the like. The lifestyle habits regarding exercise include an exercise period, menus of exercise, and the like. The lifestyle habit includes a sleep, a bath, a job, and the like. The lifestyle habit is a concept further including what is called a life environment, such as every internal environment and every external environment in life.

The attribute information of the patient includes information, such as an age, a sex, an occupation, presence/absence of a housemate, and the like, whether a relative affected with the similar symptom exists within the second degree, or results of a medical examination by medical institution.

The prescription includes every prescription to reduce the symptom, such as which nutrient is ingested, which lifestyle habit is made, and which medical agent should be ingested. Additionally, the prescription includes medical treatment surgery. As the prescription, a schedule for the prescription to be taken and a treatment plan may be presented.

The disease includes, for example, a lifestyle-related disease (high blood pressure, diabetes, dyslipidemia, and like) and every disease other than these. Pollinosis needs not be included in the disease. The disease includes an allergic symptom. The following example is described with the lifestyle-related disease as an example of this disease.

In the example of FIG. 8, a node R, which is a combination of “less than 200 (mg/dl) of a blood glucose level after two hours of drinking of 75 grams of glucose” as the symptom of the lifestyle-related disease, “less than 60 g of an intake of green and yellow vegetables” as the lifestyle habit (ingesta), and thirties to forties as the age in the attribute, a node S, which is a combination of 6.5% or more of hemoglobin in the symptom and 120 g or more of the ingestion of green and yellow vegetables in the lifestyle habit (ingesta), and a node T, which is a combination of less than 126 (mg/dl) of fasting blood glucose level in the symptom and the age of fifties or more, are each provided. In the node R, the first degree of association of “eat taking time at fixed time” is 70%, and the first degree of association of “avoid sweets and greasy meals” is 20%. In the node S, the first degree of association of “take 120 g or more of green and yellow vegetables” is 70%, the first degree of association of “eat taking time at fixed time” is 50%, and the first degree of association of “avoid sweets and greasy meals” is 30%. In the node T, the first degree of association of “take food product containing dietary fiber” is 60%, and the first degree of association of “exercise for three minutes or more/day” is 40%.

The first degree of associations of these combinations are preliminarily obtained. Next, the search unit 27 refers to the first degree of associations and determines whether the symptom, the lifestyle habit, and the attribute information newly input via the operating unit 25 correspond to which one on the left side of the first degree of association. Assume that when the symptom is “less than 200 (mg/dl) of a blood glucose level after two hours of drinking of 75 grams of glucose, the ingesta is “less than 60 g of the intake of green and yellow vegetables,” and the age in the attribute is thirties to forties, which are newly input via the operating unit 25, these correspond to the node R. Therefore, “eat taking time at fixed time” with the first degree of association of 70% and “avoid sweets and greasy meals” with the first degree of association of 20% in the node R, and the like are selected. Similarly, when the symptom is 6.5% or more of hemoglobin and the ingesta is 120 g or more of the ingestion of green and yellow vegetables, which are newly input via the operating unit 25, these correspond to the node S. Therefore, “take 120 g or more of green and yellow vegetables” with the first degree of association of 70%, “eat taking time at fixed time” with the first degree of association of 50%, “avoid sweets and greasy meals” with the first degree of association of 30% in the node S, or the like is selected.

The first degree of association may be constituted by a model that can be updated through what is called machine learning and may be constituted by a neural network. Alternatively, the first degree of association may be constituted by a network on the premise of deep learning.

The search unit 27 thus refers to the first degree of association stored in the database 3 and determines that a symptom, a lifestyle habit, and attribute information newly input via the operating unit 25 each correspond to which symptom, lifestyle habit, and attribute information arrayed on the left side of the first degree of associations.

The search unit 27 refers to these first degree of associations and executes a work to select the prescription based on the symptom, the lifestyle habit, and the attribute information newly obtained via the operating unit 25. At this time, the search unit 27 may select the prescription with the highest first degree of association. Because, as described above, the higher the first degree of association is, appropriateness of the selection becomes high. However, the search unit 27 is not limited to the case where the prescription with the highest first degree of association is selected, a prescription with the medium first degree of association or a prescription with the low first degree of association may be purposely selected. Except for these, it is obvious that a prescription with the first degree of association of 0% where a symptom, a lifestyle habit, and attribute information and a prescription are not connected with an arrow may be selected. The search unit 27 is not limited to the case of selecting one prescription but may purposely select a plurality of prescriptions with reference to the first degree of associations. The prescription searched by the search unit 27 is displayed via the display unit 23.

Note that the respective items of the symptoms, the lifestyle habits, the attribute information, and the prescriptions included in FIG. 8 are one example, and it is obvious that the above-described process operation may be executed as long as the items correspond to the symptom, the lifestyle habit, the attribute information, and the prescription.

That is, the prescription search system 1 to which the present invention is applied allows determining the prescription from the symptom newly obtained via the operating unit 25 with reference to the above-described first degree of associations. Moreover, the prescription search system 1 to which the present invention is applied can automatically execute these determination operations without manpower. This eliminates the need for taking a labor cost and time required for staff having professional knowledge performing analysis on the newly obtained symptom.

The prescription search system 1 to which the present invention is applied has a feature in searching the prescription via the first degree of associations set to three or more levels. While the first degree of association can be described by a value, for example, from 0 to 100%, the first degree of association is not limited to this. As long as the first degree of association can be described by three or more levels of values, the first degree of association may be configured into any level.

By the search based on the first degree of associations expressed by three or more levels of values, executing the search and display in an order of the high degree of associations under a condition where a plurality of prescriptions are selected is possible. When thus the first degree of associations can be displayed in the order of the high first degree of associations for the user, a prescription with a higher possibility can be preferentially selected and displayed. Meanwhile, even the prescription with the low first degree of association, a prescription specified from that can be displayed in the meaning of a second opinion, thereby ensuring providing usefulness in a case where a symptom is not improved by a prescription displayed as a first pinion or a similar case.

In addition, the present invention allows determination without missing a prescription with the extremely low first degree of association, such as 1%. This allows calling user's attention to a fact that even the prescription with the extremely low first degree of association, the prescription is related as a slight sign and therefore there may be a case where the prescription is useful as a beneficial prescription once in several ten times or several hundred times.

Further, according to the present invention, the search based on the three or more levels of the first degree of associations is advantageous in that search policy can be determined by way of setting a threshold. While decreasing the threshold allows picking up even the prescription with the above-described first degree of association of 1% without missing, there is a possibility that many prescriptions based on the first degree of associations having a low possibility of correct solution are picked up. While increasing the threshold allows narrowing down prescriptions only to prescriptions having a high possibility of correct solution, there may be a case where a prescription suggesting a preferred solution is missed once in several ten times or several hundred times. While which one is regarded as important can be determined based on a way of thinking by a user side or a system side, a degree of freedom for selection of the point regarded as important can be increased.

Further, with the present invention, the above-described first degree of associations may be updated. That is, the symptoms and the prescriptions as depicted in FIG. 8 are updated as needed. This update may be executed by reflecting the information provided via a public telecommunication network, for example, the Internet. Alternatively, the system side or the user side may artificially or automatically update the first degree of associations based on contents of, for example, research data and a thesis by an expert, a presentation at a conference, a newspaper article, and a book. An artificial intelligence may be used for these update processes.

The first degree of association is updated by increasing or decreasing the first degree of association whenever information regarding a relation between a symptom and a prescription for the symptom is input. For example, when it can be newly confirmed that a certain prescription is effective to a certain symptom through a thesis, a presentation at a conference, other research data based on experimental verification, and the like, the degree of association between the symptom and the prescription is increased. Alternatively, when it can be newly confirmed that a certain prescription is not effective to a certain symptom through a thesis, a presentation at a conference, other research data based on experimental verification, and the like, the first degree of association between the symptom and the prescription is decreased.

The first degree of associations are set to the three or more levels as described above, and this allows freely handling the first degree of association when the first degree of association is desired to increase and decrease. The update of the first degree of association itself may be executed through the above-described machine learning and deep learning.

Further, in a case where a new symptom that has not existed up to the present has been found or in a case where a new prescription that has not existed up to the present has been found, a new first degree of association may be set between them. The first degree of association of these new symptom and prescription may be updated as described above.

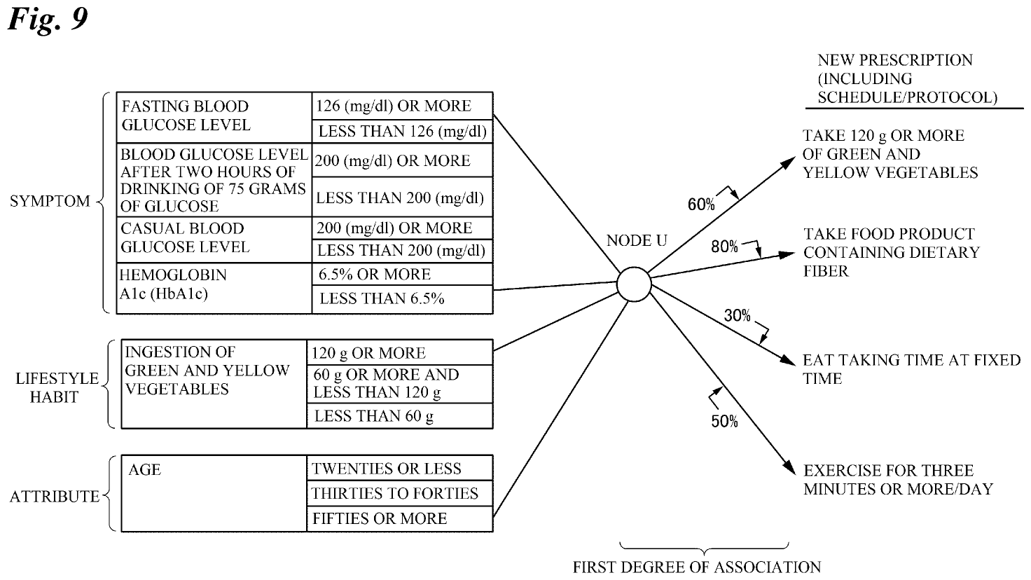

Note that the first degree of association is not limited to the above-described embodiments. An example of FIG. 9 defines the first degree of association to the node U to the combination of the plurality of symptoms. That is, to the node U, two or more of fasting blood glucose level, “blood glucose level after two hours of drinking of 75 grams of glucose,” casual blood glucose level, and hemoglobin are connected. Further, the lifestyle habit (ingesta) and the attribute are connected to this node U. New prescriptions for the node U are each associated through the first degree of associations.

Incidentally, except for the symptom, a combination of two or more lifestyle habits (ingesta) and a combination of two or more pieces of attribute information may be associated with the node.

Additionally, according to the present invention, the database 3 preliminarily storing three or more levels of the second degree of associations of the prescriptions previously taken and the respective changed symptoms of the patient who has taken the prescriptions with new prescriptions may be referred. The second degree of association is constituted by, for example, a neural network.

Here, the previously taken prescription includes any previously taken prescription on the patient, regardless of whether the prescription is the prescription searched through the prescription search system 1 to which the present invention is applied. As long as the item relates to the meal and the lifestyle habit to improve pollinosis, any item is included in the previously taken prescription. As other prescriptions previously taken, a name of a prescription medicine, an operation, a name of the ingested supplement, a frequency of ingestion, an improved lifestyle state, and the like are included.

The changed symptom here means a symptom changed through taking the prescription. The changed symptom includes a symptom worsen or a symptom not changed at all compared with the previous symptom, in addition to a symptom improved compared with a symptom before taking the prescription.

The previously taken prescription and each changed symptom of the patient who has taken the prescription are input via the operating unit 25. The input information is transmitted to the search unit 27 and the database 3.

The search unit 27 searches for a new prescription for the patient based on the information transmitted from the operating unit 25. In a process of searching, the search unit 27 refers to the information stored in the database 3.

The database 3 preliminarily stores the above-described second degree of associations. The second degree of association indicates a degree of which new prescription is highly related to a combination of each previously taken prescription and each changed symptom arrayed on the left side. In other words, the second degree of association is an index indicating that a combination of each symptom previously taken and each changed symptom is the most likely to be associated with which new prescription and indicating appropriateness to select an optimal prescription from the symptom.

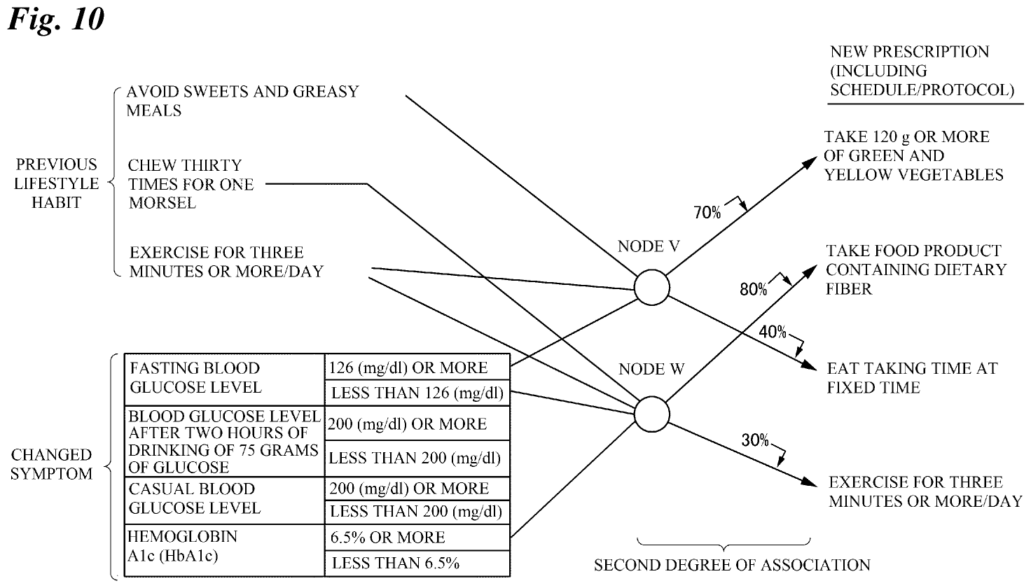

FIG. 10 illustrates an example of the second degree of associations. The respective prescriptions previously taken and the respective changed symptoms are arrayed on the left side via the second degree of associations, and the respective new prescriptions are arrayed on the right side via the second degree of associations.

In the example of FIG. 10, a node V, which includes “avoid sweets and greasy meals” and “exercise for three minutes or more/day” as the prescriptions previously taken and 126 (mg/dl) or more of fasting blood glucose level as the changed symptom, and a node W, which includes “chew thirty times for one morsel” and “exercise three minutes or more/day” as the prescriptions previously taken and less than 126 (mg/dl) of fasting blood glucose level and 6.5% or more of hemoglobin as the changed symptoms, are each provided. In the node V, the second degree of association of “take 120 g or more of green and yellow vegetables” is 70% and the second degree of association of “eat taking time at fixed time” is 40%, which are new prescriptions. In the node W, the second degree of association of “take food product containing dietary fiber” is 80%, and the second degree of association of “exercise for three minutes or more/day” is 30%, which are new prescriptions.

The second degree of associations of these combinations are preliminarily obtained. Next, the search unit 27 refers to the second degree of associations and determines whether the previously taken prescription and each changed symptom of the patient who has taken the prescription, which are newly input via the operating unit 25, each correspond to which item arrayed on the left side of the second degree of associations. Assume that when the prescriptions previously taken input via the operating unit 25 are “chew thirty times for one morsel” and “exercise for three minutes or more/day” and the changed symptoms are less than 126 (mg/dl) of fasting blood glucose level and 6.5% or more of hemoglobin, these correspond to the node W. In this case, with reference to the second degree of associations of the node W, as new prescriptions, “take food product containing dietary fiber,” “exercise for three minutes or more/day,” or the like is selected.

Thus, a new prescription can be automatically searched based on the previously taken prescription and each changed symptom of the patient who has taken the prescription. This allows continuously observing the symptom of the patient and an optimal prescription according to the symptom can be selected. In a case where the previously taken prescription is comparatively strong and the symptom of the patient is improved after that, the best prescription can be continuously proposed timely, such as switching the prescription to a prescription less effective compared with the previous prescription.

Incidentally, the second degree of association may be similarly updated. That is, the previously taken prescription, each changed symptom, and the new prescription as depicted in FIG. 10 are updated as needed. This update may be executed by reflecting the information provided via a public telecommunication network, for example, the Internet. Alternatively, the system side or the user side may artificially or automatically update the second degree of associations based on contents of, for example, research data and a thesis by an expert, a presentation at a conference, a newspaper article, and a book. An artificial intelligence may be used for these update processes.

Obviously, the second degree of association may associate between a combination of one or more of any of the above-described ingesta, attribute information, lifestyle habit, character, and external information, in addition to the previously taken prescription and each changed symptom, and the prescription.

Further, the process operation of the second degree of association is not limited to the case where the previously taken prescription found by the search based on the above-described process operation of the first degree of association and the symptom changed based on the prescription are input, but a previously taken prescription regardless of this may be input. In this case, by further enlarging a range more than that of the previous prescription, the reference may be executed including the previous lifestyle habits. The previous lifestyle habits may include history information of the lifestyle habits up to the present or may be lifestyle habits intermittently sensed previously at a certain time point. Similarly, the changed symptom may include a change history of the symptom up to the present or may be a symptom intermittently sensed previously at a certain time point.

### Search for Prescription of Hair Growth

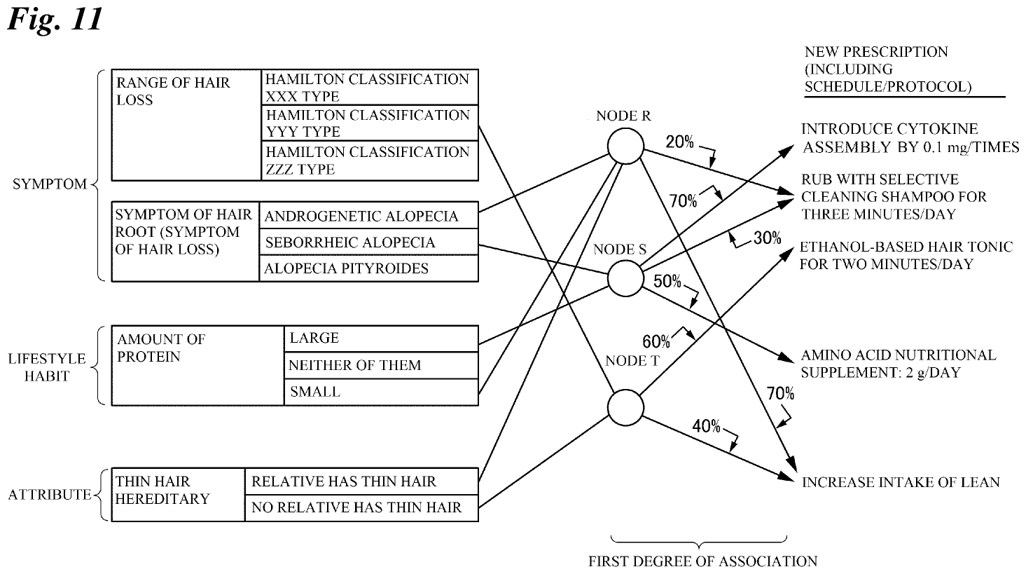

FIG. 11 illustrates an example in which three or more levels of the first degree of associations are preliminarily defined between respective symptoms of thin hair and prescription for a patient. The respective symptoms are arrayed on the left side via the first degree of associations, and the respective prescriptions are arrayed on the right side via the first degree of associations. The first degree of association indicates a degree that which prescription has a high relevance to the symptom arrayed on the left side.

In addition to these, the first degree of association associates one or more prescriptions with a combination of each symptom, a lifestyle habit, and attribute information.

The symptoms are various symptoms of thin hair and includes, for example, a direct symptom, such as a range of hair loss, a symptom of hair root, and the number of fallen hair, and an indirect symptom, such as a state of scalp and an amount of minerals inside of a body. These symptoms include indexes expressed in various medical treatment data, results (including a VAS evaluation) sensed by a doctor, the patient, an estimator, and the like as sensed values, and the like.

The lifestyle habit includes all events related to the life of the patient. Examples of the lifestyle habit regarding an eating habit include the ingesta and an amount of a meal ingested during the meal, time of the meal, and the like. The ingesta here include any kind of ingesta ingestible by the patient, such as food, a beverage, a supplement, and a medical agent. The lifestyle habits regarding a sleep include hours of sleep, hour of rising, bedtime, and the like. The lifestyle habits regarding exercise include an exercise period, menus of exercise, and the like. The lifestyle habits regarding hair-care include a care of hair and a scalp with a shampoo, a hair tonic, and the like, measures to prevent ultraviolet rays, a massage, and the like.

The attribute information of the patient includes information, such as an age, a sex, an occupation, presence/absence of a housemate, and the like, whether a relative affected with the similar symptom exists within the second degree, or results of a medical examination by medical institution.

The prescription includes every prescription to reduce the symptom, such as which nutrient is ingested, which lifestyle habit is made, and which medical agent should be ingested. Additionally, the prescription includes medical treatment surgery. As the prescription, a schedule for the prescription to be taken and a treatment plan may be presented.

In the example of FIG. 11, a node R, which is a combination of a case where a symptom of hair root in a symptom of thin hair is androgenetic alopecia, an amount of protein in a lifestyle habit (ingesta) is small, and relative has thin hair in thin hair hereditary in attribute information, a node S, which is a combination of a case where the symptom of hair root in the symptom is seborrheic alopecia and the amount of protein in the lifestyle habit (ingesta) is large, and a node T, which is a combination of a case where the range of hair loss in the symptom is the Hamilton classification YYY and no relative has thin hair in the thin hair hereditary in the attribute information, are each provided. In the node R, the first degree of association of “increase intake of lean” is 70%, and the first degree of association of “rub with selective cleaning shampoo for three minutes/day” is 20%. In the node S, the first degree of association of “introduce cytokine assembly by 0.1 mg/times” is 70%, the first degree of association of “amino-acid-based nutritional supplement: 2 g/day” is 50%, and the first degree of association of “rub with selective cleaning shampoo for three minutes/day” is 30%. In the node T, the first degree of association of “ethanol-based hair tonic for two minutes/day” is 60%, and the first degree of association of “increase intake of lean” is 40%.

The first degree of associations of these combinations are preliminarily obtained. Next, the search unit 27 refers to the first degree of associations and determines whether the symptom, the lifestyle habit, and the attribute information newly input via the operating unit 25 correspond to which ones on the left side of the first degree of associations. Assume that when the symptom is the androgenetic alopecia in the symptom of hair root in the symptom of thin hair, the amount of protein in the lifestyle habit (ingesta) is small, and relative has thin hair in the thin hair hereditary in the attribute information, which are newly input via the operating unit 25, these correspond to the node R. Therefore, “increase intake of lean” with the first degree of association of 70% or “rub with selective cleaning shampoo for three minutes/day” with the first degree of association of 20% in the node R, or the like is selected.

Note that the respective items of the symptoms, the lifestyle habits, the attribute information, and the prescriptions included in FIG. 11 are one example, and it is obvious that the above-described process operation may be executed as long as the items correspond to the symptom, the lifestyle habit, the attribute information, and the prescription.

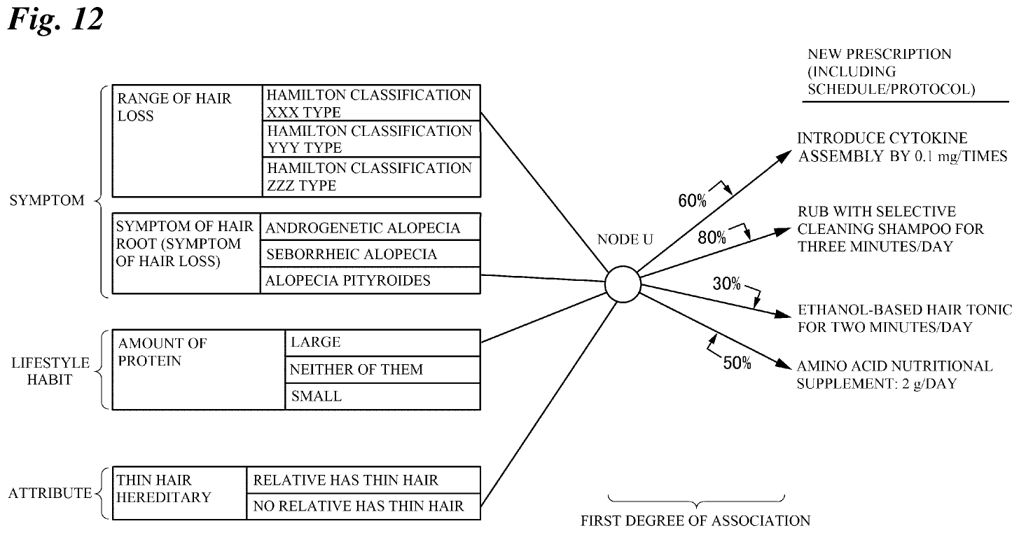

Note that the first degree of association is not limited to the above-described embodiments. An example of FIG. 12 defines the first degree of association to the node U to the combination of the plurality of symptoms. That is, to the node U, the Hamilton classification XXX type in the range of hair loss, alopecia pityroides in the symptom of hair root, large in the amount of protein in the lifestyle habit (ingesta), and no relative has thin hair in the thin hair hereditary in the attribute information are connected. That is, two or more symptoms are connected to the node U.

Incidentally, except for the symptom, a combination of two or more lifestyle habits (ingesta) and a combination of two or more pieces of attribute information may be associated with the node U.

Additionally, according to the present invention, the database 3 preliminarily storing three or more levels of the second degree of associations of the prescriptions previously taken and the respective changed symptoms of the patient who has taken the prescriptions with new prescriptions may be referred.

Note that it is not limited to what is called the lifestyle-related disease in this embodiment, and the search can be similarly executed in a case of searching for a prescription for any kind of disease.

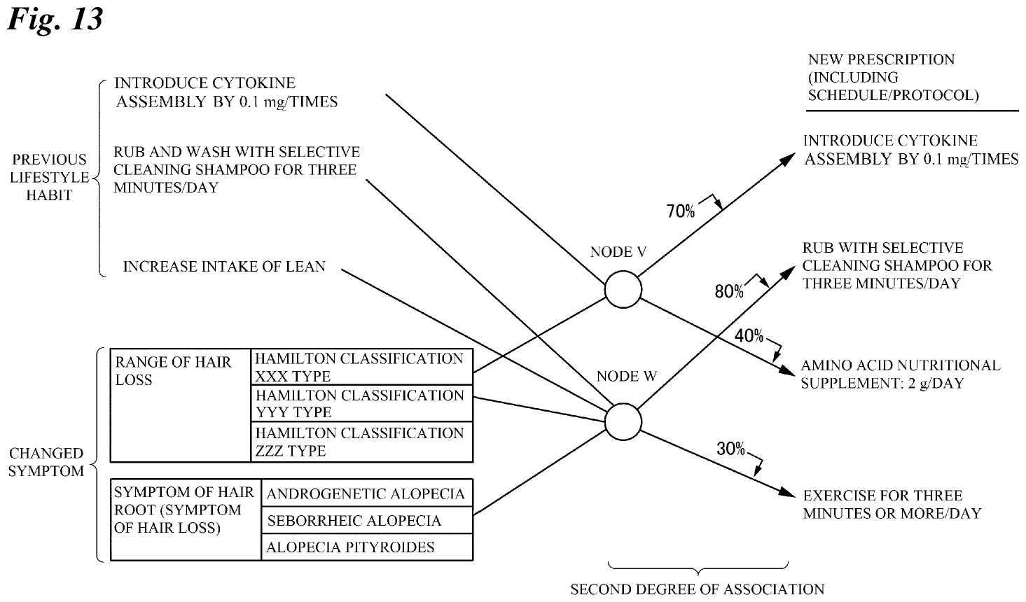

FIG. 13 illustrates an example of the second degree of associations. The respective prescriptions previously taken and the respective changed symptoms are arrayed on the left side via the second degree of associations, and respective new prescriptions are arrayed on the right side via the second degree of associations.

In the example of FIG. 13, a node V and a node W are each provided. Each of “introduce cytokine assembly by 0.1 mg/times” and “increase intake of lean” as the prescriptions previously taken and “Hamilton classification XXX type” in the range of hair loss as the changed symptom link in the node V. Each of “rub and wash with selective cleaning shampoo for three minutes/day” and “increase intake of lean” as the prescriptions previously taken and “seborrheic alopecia” in the symptom of hair root as the changed symptom link in the node W. In the node V, as the new prescriptions, the second degree of association of “introduce cytokine assembly by 0.1 mg/times” is 70%, and the second degree of association of “amino acid nutritional supplement: 2 g/day” is 40%. In the node W, as the new prescriptions, the second degree of association of “rub with selective cleaning shampoo for three minutes/day” is 80%, and the second degree of association of “exercise for three minutes or more/day” is 30%.

The second degree of associations of these combinations are preliminarily obtained. Next, the search unit 27 refers to the second degree of associations and determines whether the previously taken prescription and each changed symptom of the patient who has taken the prescription, which are newly input via the operating unit 25, each correspond to which item arrayed on the left side of the second degree of associations. Assume that the prescriptions previously taken are “rub and wash with selective cleaning shampoo for three minutes/day” and “increase intake of lean” and the changed symptoms are the Hamilton classification YYY in the range of hair loss and the seborrheic alopecia in the symptom of hair root, which are input via the operating unit 25, these correspond to the node W. In this case, with reference to the second degree of associations of the node W, as the new prescription, “rub and wash with selective cleaning shampoo for three minutes/day,” “exercise for three minutes or more/day,” or the like is selected.

Incidentally, the second degree of association may be similarly updated. That is, the previously taken prescription, each changed symptom, and the new prescription as depicted in FIG. 10 are updated as needed. This update may be executed by reflecting the information provided via a public telecommunication network, for example, the Internet. Alternatively, the system side or the user side may artificially or automatically update the second degree of associations based on contents of, for example, research data and a thesis by an expert, a presentation at a conference, a newspaper article, and a book. An artificial intelligence may be used for these update processes.

Obviously, the second degree of association may associate between a combination of one or more of any of the above-described ingesta, attribute information, lifestyle habit, character, and external information, in addition to the previously taken prescription and each changed symptom, and the prescription.

Further, the process operation of the second degree of association is not limited to the case where the previously taken prescription found by the search based on the above-described process operation of the first degree of association and the symptom changed based on the prescription are input, but a previously taken prescription regardless of this may be input. In this case, by further enlarging a range more than that of the previous prescription, the reference may be executed including the previous lifestyle habits. The previous lifestyle habits may include history information of the lifestyle habits up to the present or may be lifestyle habits intermittently sensed previously at a certain time point. Similarly, the changed symptom may include a change history of the symptom up to the present or may be a symptom intermittently sensed previously at a certain time point.

### Search for Prescription of Beauty Care

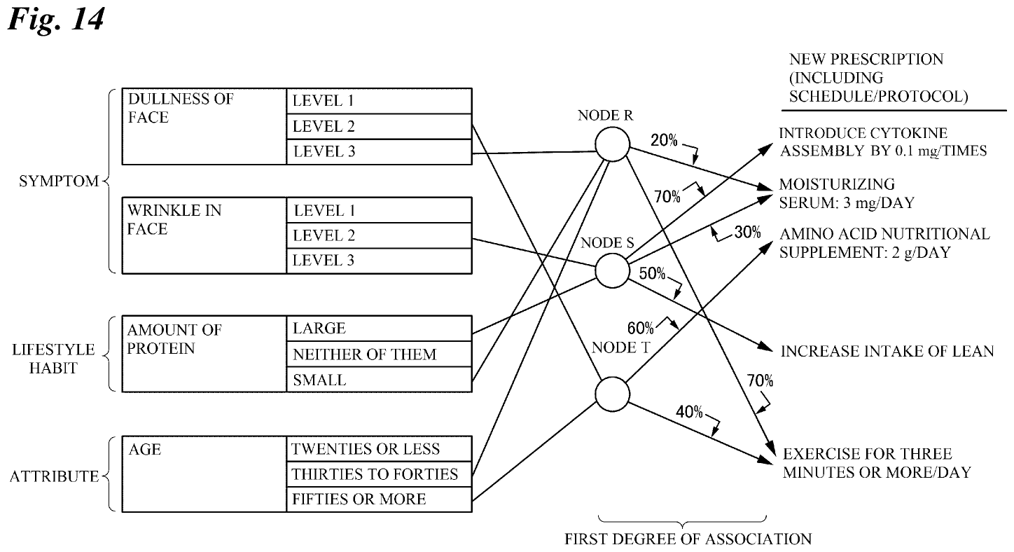

FIG. 14 illustrates an example in which three or more levels of the first degree of associations are preliminarily defined between respective symptoms regarding a beauty care and prescriptions. The respective symptoms are arrayed on the left side via the first degree of associations, and the respective prescriptions are arrayed on the right side via the first degree of associations. The first degree of association indicates a degree that which prescription has a high relevance to the symptom arrayed on the left side.

In addition to these, the first degree of association associates one or more prescriptions with a combination of each symptom, a lifestyle habit, and attribute information.

The symptoms are various symptoms related to the beauty care and includes, for example, a direct symptom, such as dullness of a face, a wrinkle in face, and a blotch on the face, and an indirect symptom, such as an amount of moisture of skin and an amount of minerals inside of a body. These symptoms include indexes expressed in various medical treatment data, results (including a VAS evaluation) sensed by a doctor, the patient, an estimator, and the like as sensed values, and the like.

The lifestyle habit includes all events related to the life of the patient. Examples of the lifestyle habit regarding an eating habit include the ingesta and an amount of a meal ingested during the meal, time of the meal, and the like. The ingesta here include any kind of ingesta ingestible by the patient, such as food, a beverage, a supplement, and a medical agent. The lifestyle habits regarding a sleep include hours of sleep, hour of rising, bedtime, and the like. The lifestyle habits regarding exercise include an exercise period, menus of exercise, and the like. The lifestyle habits regarding a skin care include a skin care with moisturizing serum, measures for preventing ultraviolet rays, cleansing, a massage, and the like.

The attribute information of the patient includes information, such as an age, a sex, an occupation, presence/absence of a housemate, and the like, whether a relative affected with the similar symptom exists within the second degree, or results of a medical examination by medical institution.

The prescription includes every prescription to reduce the symptom, such as which nutrient is ingested, which lifestyle habit is made, and which medical agent should be ingested. Additionally, the prescription includes medical treatment surgery. As the prescription, a schedule for the prescription to be taken and a treatment plan may be presented.

In an example of FIG. 14, a node R, which is a combination of a case where the dullness of face in the symptom is a level 3, the amount of protein in the lifestyle habit (ingesta) is small, and the age in the attribute is thirties to forties, a node S, which is a combination of a case where the wrinkle in face in the symptom is a level 2 and the protein in the lifestyle habit (ingesta) is large, and a node T, which is a combination of a case where the dullness of face in the symptom is a level 2 and the age in the attribute information is fifties or more, are each provided. In this node R, the first degree of association of “exercise for three minutes or more/day” is 70%, and the first degree of association of “moisturizing serum: 3 mg/day” is 20%. In the node S, the first degree of association of “introduce cytokine assembly by 0.1 mg/times” is 70%, the first degree of association of “increase intake of lean” is 50%, and the first degree of association of “moisturizing serum: 3 mg/day” is 30%. In the node T, the first degree of association of “amino acid nutritional supplement: 2 g/day” is 60%, and the first degree of association of “exercise for three minutes or more/day” is 40%.

The first degree of associations of these combinations are preliminarily obtained. Next, the search unit 27 refers to the first degree of associations and determines whether the symptom, the lifestyle habit, and the attribute information newly input via the operating unit 25 correspond to which ones on the left side of the first degree of associations. Assume that when the dullness of face in the symptom of the beauty care as the symptom is the level 3, the amount of protein in the lifestyle habit (ingesta) is small, and the age in the attribute is thirties to forties, which are newly input via the operating unit 25, these correspond to the node R. Therefore, “exercise for three minutes or more/day” with the first degree of association of 70% or “moisturizing serum: 3 mg/day” with the first degree of association of 20% in the node R, or the like is selected.

Note that the respective items of the symptoms, the lifestyle habits, the attribute information, and the prescriptions included in FIG. 14 are one example, and it is obvious that the above-described process operation may be executed as long as the items correspond to the symptom, the lifestyle habit, the attribute information, and the prescription.

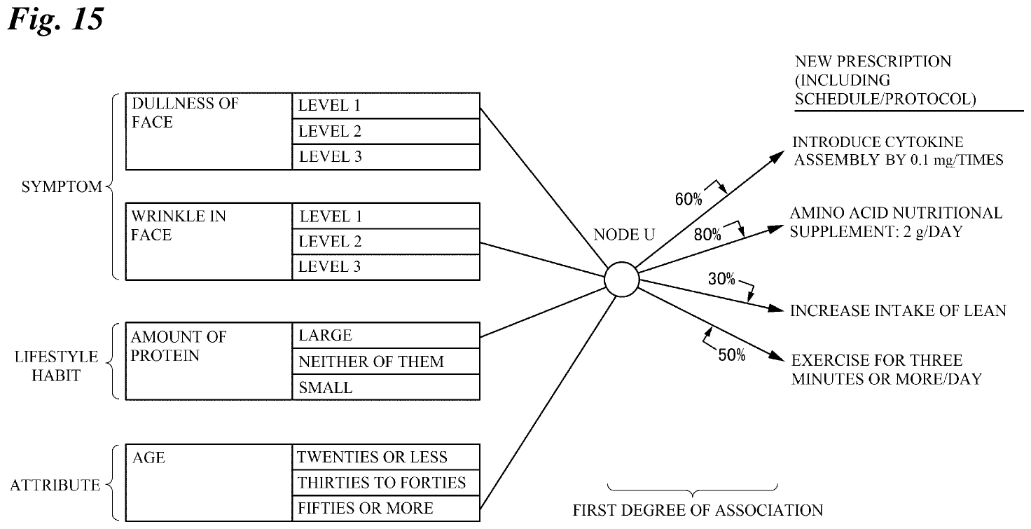

Note that the first degree of association is not limited to the above-described embodiments. An example of FIG. 15 defines the first degree of association to the node U to the combination of the plurality of symptoms. That is, to the node U, the level 1 of the dullness of face, the level 2 of the wrinkle in face, large in the amount of protein in the lifestyle habit (ingesta), and the fifties or more in the age in the attribute information are connected. That is, two or more symptoms are connected to the node U.

Incidentally, a combination of two or more lifestyle habits and a combination of two or more pieces of attribute information may be associated with the node U, except for the symptom.

Additionally, according to the present invention, the database 3 preliminarily storing three or more levels of the second degree of associations of the prescriptions previously taken and the respective changed symptoms of the patient who has taken the prescriptions with new prescriptions may be referred.

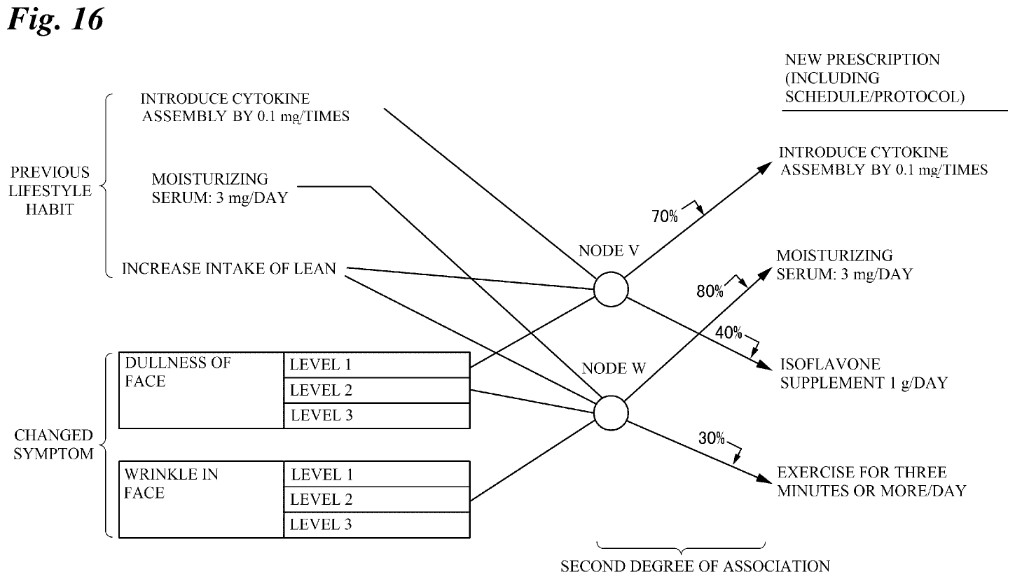

FIG. 16 illustrates an example of the second degree of associations. The respective prescriptions previously taken and the respective changed symptoms are arrayed on the left side via the second degree of associations, and respective new prescriptions are arrayed on the right side via the second degree of associations.

In the example of FIG. 16, a node V and a node W are each provided. Each of “introduce cytokine assembly by 0.1 mg/times” and “increase intake of lean” as the prescriptions previously taken, and the level 1 of the dullness of face as the changed symptom link in the node V. Each of “moisturizing serum: 3 mg/l” and “increase intake of lean” as the prescriptions previously taken, and the level 2 of the dullness of face and the level 2 of the wrinkle in face link in the node W. In the node V, as new prescriptions, the second degree of association of “introduce cytokine assembly by 0.1 mg/times” is 70%, and the second degree of association of “isoflavone supplement: 1 g/day” is 40%. In the node W, as new prescriptions, the second degree of association of “moisturizing serum: 3 mg/day” is 80%, and the second degree of association of “exercise for three minutes or more/day” is 30%.

The second degree of associations of these combinations are preliminarily obtained. Next, the search unit 27 refers to the second degree of associations and determines whether the previously taken prescription and each changed symptom of the patient who has taken the prescription, which are newly input via the operating unit 25, each correspond to which item arrayed on the left side of the second degree of association. Assume that when the prescriptions previously taken are “moisturizing serum: 3 mg/l” and “increase intake of lean” and the changed symptoms are the level 2 in the dullness of face and the level 2 in the wrinkle in face, which are input via the operating unit 25, these correspond to the node W. In this case, with reference to the second degree of associations of the node W, as a new prescription, “moisturizing serum: 3 mg/day,” “exercise for three minutes or more/day,” or the like are selected.

Incidentally, the second degree of association may be similarly updated. That is, the previously taken prescription, each changed symptom, and the new prescription as depicted in FIG. 10 are updated as needed. This update may be executed by reflecting the information provided via a public telecommunication network, for example, the Internet. Alternatively, the system side or the user side may artificially or automatically update the second degree of associations based on contents of, for example, research data and a thesis by an expert, a presentation at a conference, a newspaper article, and a book. An artificial intelligence may be used for these update processes.

Obviously, the second degree of association may associate between a combination of one or more of any of the above-described ingesta, attribute information, lifestyle habit, character, and external information, in addition to the previously taken prescription and each changed symptom, and the prescription.

Further, the process operation of the second degree of association is not limited to the case where the previously taken prescription found by the search based on the above-described process operation of the first degree of association and the symptom changed based on the prescription are input, but a previously taken prescription regardless of this may be input. In this case, by further enlarging a range more than that of the previous prescription, the reference may be executed including the previous lifestyle habits. The previous lifestyle habits may include history information of the lifestyle habits up to the present or may be lifestyle habits intermittently sensed previously at a certain time point. Similarly, the changed symptom may include a change history of the symptom up to the present or may be a symptom intermittently sensed previously at a certain time point.

What is claimed is:

1. A non-transitory computer-readable recording medium storing allergy prescription search program which causes a computer to execute:
an input step of inputting an actual symptom of an allergy;
a search step of referring to three or more levels of first degree of associations between each symptom of an allergy and a prescription for the symptom stored in a database to search for one or more prescriptions based on the symptom input in the input step;
a second degree of association obtaining step of obtaining three or more levels of second degree of associations of a previously taken prescription found by the search in the search step and each improved symptom of a patient who has taken the prescription with a new prescription;
an improved symptom input step of inputting an improved symptom of the patient who has actually taken the previously taken prescription; and
a new prescription search step of referring to the second degree of associations obtained in the second degree of association obtaining step to search for one or more new prescriptions based on the improved symptom input in the improved symptom input step and the previously taken prescription.

2. The non-transitory computer-readable recording medium storing allergy prescription search program according to claim 1, comprising
an updating step of reflecting a relation between the previously taken prescription and each of the improved symptoms of the patient who has taken the prescription, and the new prescription, to the second degree of association to update the second degree of association when the updating step obtains the relation.

3. The non-transitory computer-readable recording medium storing allergy prescription search program according to claim 2, wherein
the updating step executes the reflection to the second degree of association constituted by a neural network to update the second degree of association through utilization of an artificial intelligence.

4. A non-transitory computer-readable recording medium storing prescription search program which causes a computer to execute:
a first degree of association obtaining step of preliminarily obtaining three or more levels of first degree of associations of a combination of each symptom and any one or more of each lifestyle habit and each piece of attribute information with a prescription for the symptom;
an input step of inputting information including the combination;
a search step of referring to the first degree of associations obtained by the first degree of association obtaining step to search for one or more prescriptions based on the information input in the input step;
a second degree of association obtaining step of obtaining three or more levels of second degree of associations of a previously taken prescription found by the search in the search step and each changed symptom of a patient who has taken the prescription with a new prescription;
a changed symptom input step of inputting an actual changed symptom of the patient who has taken the previously taken prescription; and
a new prescription search step of referring to the second degree of associations obtained in the second degree of association obtaining step to search for one or more new prescriptions based on the changed symptom input in the changed symptom input step and the previously taken prescription.

5. A non-transitory computer-readable recording medium storing prescription search program which causes a computer to execute respective steps of:
a second degree of association obtaining step of obtaining three or more levels of second degree of associations of a previous lifestyle habit and each changed symptom changed from previously with a new prescription;
a changed symptom input step of inputting an actual previous lifestyle habit and an actual changed symptom of a patient who has made the lifestyle habit; and
a new prescription search step of referring to the second degree of associations obtained in the second degree of association obtaining step to search for one or more new prescriptions based on the previous lifestyle habit and the changed symptom input in the changed symptom input step.

6. The non-transitory computer-readable recording medium storing prescription search program according to claim 4, comprising
an updating step of reflecting a relation between the previously taken prescription and each of the improved symptoms of the patient who has taken the prescription, and the new prescription, to the second degree of association to update the second degree of association when the updating step obtains the relation.

7. The non-transitory computer-readable recording medium storing prescription search program according to claim 6, wherein
the updating step executes the reflection to the second degree of association constituted by a neural network to update the second degree of association through utilization of an artificial intelligence.

8. The non-transitory computer-readable recording medium storing prescription search program according to claim 5, comprising
an updating step of reflecting a relation between the previously taken prescription and each of the improved symptoms of the patient who has taken the prescription, and the new prescription, to the second degree of association to update the second degree of association when the updating step obtains the relation.

9. The non-transitory computer-readable recording medium storing prescription search program according to claim 8, wherein
the updating step executes the reflection to the second degree of association constituted by a neural network to update the second degree of association through utilization of an artificial intelligence.
